(19) United States
(12) Patent Application Publication
(10) Pub. No.: US 2026/0137745 A1
(43) Pub. Date: May 21, 2026

(54) QUERCETAGETIN ENRICHED MARIGOLD EXTRACTS, COMPOSITIONS, METHODS OF PREPARATION AND USE THEREOF

(71) Applicant: AVT NATURAL PRODUCTS LTD., Chennai, Tamil Nadu (IN)

(72) Inventors: Rahul THOMAS, Aluva, Kerala (IN); Subarna Saha DHAR, Aluva, Kerala (IN); Ezhil Arasan RAMANAN, Aluva, Kerala (IN); Naijo VARGHESE, Aluva, Kerala (IN); Abhijith Kunhitlu SUBRAHMANYA, Aluva, Kerala (IN)

(21) Appl. No.: 19/122,526

(22) PCT Filed: Oct. 17, 2023
(86) PCT No.: PCT/IN2023/050952

§ 371 Date: Apr. 18, 2025

(30) Foreign Application Priority Data
Oct. 21, 2022 (IN) 202241060193

(51) Int. Cl. A61K 36/28; A61K 8/49; A61K 8/63; A61K 8/9789; A61K 31/352; A61K 31/575; A61P 17/02; A61P 17/08; A61P 17/10; A61P 17/18; A61P 29/00; A61Q 19/00; A61Q 19/02

(52) U.S. Cl. CPC A61K 36/28; A61K 8/498; A61K 8/63; A61K 8/9789; A61K 31/352; A61K 31/575; A61P 17/02; A61P 17/08; A61P 17/10; A61P 17/18; A61P 29/00; A61Q 19/007; A61Q 19/008; A61Q 19/02; A61K 2236/333; A61K 2236/35; A61K 2236/39; A61K 2236/53; A61K 2800/522; A61K 2800/782; A61K 2800/805

(57) ABSTRACT

The present disclosure provides extracts and compositions comprising quercetagetin. In particular, the extracts are marigold extracts enriched with quercetagetin for cosmeceutical and dermatological application. Accordingly, embodiments herein include a cosmetic method and a dermatological method of treating skin by applying or administering, topically, the compositions comprising the marigold extracts disclosed herein.

### FIELD OF INVENTION

The present disclosure relates to extracts and compositions comprising quercetagetin, and method of preparation thereof. Further, the present disclosure relates to a method of treating skin using the extracts and compositions comprising quercetagetin.

### BACKGROUND OF INVENTION

Quercetagetin, also known as 6-Hydroxyquercetin, is a flavonoid which is structurally similar to quercetin. While both, quercetin and quercetagetin have the same basic flavanol structure having two phenyl rings (A and B) and a heterocyclic ring (C), quercetagetin includes an additional OH group at the C6 position of ring-A of the flavanol structure.

Quercetagetin is a flavonoid present in flowers of Marigold. Marigold plant (Tagetes erecta Linn) is grown all over the world as an ornamental plant and has traditional medicine application in China and Mexico. It is commercially grown in India, China and Mexico mainly for the purpose of extracting lutein. There are few studies published over the decade on Quercetagetin's anti-inflammatory, antioxidant and co pigment activities (Biomol Ther. 2013; 21:138-145; Fitoterapia. 2012; 83:481-489 and Food Chem. 2015; 166:50-55).

Flavonoid compositions for improving the appearance of scar tissue and other topical applications, to some extent, are known (refer U.S. Pat. No. 7,399,783 B2, EP2034977B1, KR20120035741A, Nissha Bharathi et al (Biotechnology & Biotechnological Equipment, Volume 35, 2021-Issue 1, etc). One such composition is demonstrated with, quercetin dihydrate. Hence, topical compositions comprising flavonoids are known for treatment of skin.

Particularly, melanin inhibition has been known to achieve even skin tone and skin brightening, and increased Tyrosinase activity achieves increase in melanin production. Hence, it is desirable that skin treatment formulations also exhibit improved melanin and tyrosinase inhibition to achieve skin brightening and even skin tone. While the effect of melanin and tyrosinase inhibition on skin is known (refer Ombon Vallisuta et al in Experimental and Therapeutical Medicine 7, 246-250, 2014; and Pimporn Leelapornpisid et al in International Journal of Pharmacy and Pharmaceutical Sciences Vol 6, Issue 2, 2014). While various skin care compositions may be known, there exists a need for holistic approaches of skin care.

### SUMMARY OF THE INVENTION

In an aspect of the present disclosure, there is provided a method for preparing a marigold extract, said method comprising: (a) subjecting marigold biomass to extraction using an alcohol selected from ethanol, methanol, or combination thereof, to obtain a miscella fraction; and (b) evaporating the miscella fraction to obtain the marigold extract.

In an aspect of the present disclosure, there is provided a method for preparing a marigold extract, said method comprising: (a) subjecting marigold biomass to extraction using an alcohol selected from ethanol, methanol, or combination thereof, to obtain a miscella fraction; (b) evaporating the miscella fraction to obtain a first extract; (c) treating the first extract with a solvent comprising n-hexane to obtain a residue; and (d) drying the residue to obtain the marigold extract.

In an aspect of the present disclosure, there is provided a method for preparing a marigold extract enriched with quercetagetin, said method comprising: (a) subjecting marigold biomass to extraction using an alcohol selected from ethanol, methanol, or combination thereof, to obtain a miscella fraction; (b) evaporating the miscella fraction to obtain a first extract; (c) treating the first extract with a solvent comprising n-hexane to obtain a residue; (d) drying the residue to obtain a second extract; (e) enriching by treating the second extract with ethanol and activated carbon; and (f) removing the activated carbon, preferably by filtering, and ethanol, preferably by distillation, to obtain the quercetagetin enriched marigold extract.

In an aspect of the present disclosure, there is provided a marigold extract enriched with quercetagetin for cosmetic and dermatologic treatment of skin, comprising quercetagetin in an amount ranging from at least 40 wt % to 99 wt %; patuletin in an amount ranging from 1 wt % to 7 wt %; 6-Hydroxykaempherol in an amount ranging from 0 wt % to 4 wt %; and phytosterols in an amount ranging from 0 wt % to 2.5 wt %.

In an aspect of the present disclosure, there is provided a topical composition for cosmetic and dermatologic treatment of skin, said composition comprising the marigold extract in an amount ranging from at least 0.1 wt % to 99 wt %, in respect of the topical composition; and at least one cosmetically acceptable carrier.

In an aspect of the present disclosure, there is provided a method for preparing the topical composition for cosmetic and dermatologic treatment of skin, said method comprising: blending the marigold extract and the cosmetically acceptable carrier to obtain the composition.

In an aspect of the present disclosure, there is provided a cosmetic method comprising applying the topical composition on a target site on skin to decrease sebum secretion, reduce inflammation, improve skin moisturization, control trans epidermal water loss, achieve skin brightening, and/or maintain even tone of skin, at the target site.

In an aspect of the present disclosure, there is provided a dermatological method for treatment of skin, comprising administering, topically, the topical composition on a target site on the skin to elicit 5α-reductase inhibition, anti-inflammatory activity, and/or anti-acne property at the target site.

In an aspect of the present disclosure, there is provided a method for preparing a marigold extract enriched with quercetagetin, said method comprising: (a) extracting marigold biomass using an alcohol selected from ethanol and methanol, to obtain a miscella fraction, wherein the ratio of biomass extract and alcohol is in the range of 1:3 to 1:5; (b) evaporating the miscella fraction to obtain a first extract comprising quercetagetin in the range of at least 40-55 wt % in respect of the first extract; (c) optionally repeating step (a) and (b); (d) treating the first extract with a solvent comprising n-hexane to obtain a residue, wherein the ratio of first extract and solvent is in the range of 1:3 to 1:5; (e) drying the residue to remove the solvent and obtain a second extract comprising quercetagetin in the range of at least 60-75 wt % in respect of the second extract; (f) optionally repeating step (d) and (e); (g) enriching by treating the second extract with ethanol and activated carbon, wherein the ratio of second extract and ethanol is in the range of 1:30 to 1:50; (h) removing the activated carbon, preferably by filtering, and ethanol, preferably by distillation, to obtain the marigold extract enriched with quercetagetin comprising quercetagetin in an amount of at least 90 wt %; and (i) optionally repeating step (g) and (h) to obtain the marigold extract.

These and other features, aspects, and advantages of the present subject matter will be better understood with reference to the following description and appended claims. This summary is provided to introduce a selection of concepts in a simplified form. This summary is not intended to identify key features or essential features of the claimed subject matter, nor is it intended to be used to limit the scope of the claimed subject matter.

### BRIEF DESCRIPTION OF DRAWINGS

The following drawings form a part of the present specification and are included to further illustrate aspects of the present disclosure. The disclosure may be better understood by reference to the drawings in combination with the detailed description of the specific embodiments presented herein.

FIG. 1 depicts results of DPPH Antioxidant Assays, Oxygen Radical Absorbance Capacity assay, and Cellular ROS Assay for the composition comprising QG40, and composition comprising QG90, and composition having quercetin, according to embodiments herein;

FIG. 2 depicts anti-inflammatory effect of the composition comprising QG90 extract on LPS induced IL-1A & TNF-alpha release in THP-1 cells, according to embodiments herein;

FIG. 3 depicts inhibitory effect of compositions comprising QG40 against LPS induced IL-6, IL-1A & TNF-alpha release in THP-1 cells, according to embodiments herein;

FIG. 4 depicts inhibitory effect of composition comprising QG90 extract against Ultraviolet Radiation (UVR) induced IL-6, IL-1A & TNF-alpha release in THP-1 cells, according to embodiments herein;

FIG. 5 depicts inhibitory effect of composition comprising QG40 against Ultraviolet Radiation (UVR) induced IL-6, IL-1A & TNF-alpha release in THP-1 cells, according to embodiments herein;

FIG. 6 depicts cellular melanin inhibition assay in human adult epidermal melanocytes composition comprising QG90, composition comprising QG40, and composition having quercetin, according to embodiments herein; and

FIG. 7 depicts the effect of QG90 (Example 1) formulation on the skin tone improvement over 28 days' time, according to embodiments herein.

### DESCRIPTION OF THE INVENTION

Those skilled in the art will be aware that the present disclosure is subject to variations and modifications other than those specifically described. It is to be understood that the present disclosure includes all such variations and modifications. The disclosure also includes all such steps, features, compositions, and compounds referred to or indicated in this specification, individually or collectively, and any and all combinations of any or more of such steps or features.

### Definitions

For convenience, before further description of the present disclosure, certain terms employed in the specification and examples are delineated here. These definitions should be read in the light of the remainder of the disclosure and understood by a person of skill in the art. The terms used herein have the meanings recognized and known to those of skill in the art. However, for convenience and completeness, particular terms and their meanings are set forth below.

The articles “a”, “an” and “the” are used to refer to one or to more than one (i.e., to at least one) of the grammatical objects of the article.

Throughout this specification, unless the context requires otherwise, the word “comprise” and variations such as “comprises” and “comprising” are used in the inclusive, open sense and will be understood to imply the inclusion of a stated element or step or group of elements or steps but not the exclusion of any other element or step or group of elements or steps. It is not intended to be construed as “consists of only”.

The term “including”, as used herein, means “including but not limited to”. “Including” and “including but not limited to” are used interchangeably.

Embodiments herein provide extracts and compositions comprising quercetagetin. In particular, the extracts as disclosed herein are marigold extracts comprising quercetagetin. Marigold, according to the present disclosure, refers to a plant of the genus Tagetes, particularly Tagetes erecta. In an embodiment, marigold refers to the flower of the plant of the genus Tagetes. Accordingly, the term marigold extract refers to the extract of flowers of marigold. Marigold flowers include various natural compounds such as terpenes, carotenoids and flavonoids.

The marigold extract, according to the present disclosure, comprises quercetagetin as the main flavanoid. In general, marigold flowers are used to extract lutein which is a commercially valuable product. The present inventors have observed that the quercetagetin, particularly the marigold extract comprising quercetagetin, are cosmetically and dermatologically effective. Accordingly, embodiments herein include a cosmetic method and a dermatological method of treating skin by applying or administering, topically, the compositions comprising the marigold extracts as disclosed herein. Embodiments herein are capable of decreasing sebum secretion, reducing inflammation, improving skin moisturization, controlling trans epidermal water loss, eliciting 5α-reductase inhibition, anti-acne activity and/or skin brightening activity at the target site. Embodiments herein are capable of maintaining even skin tone. It is observed that the embodiments herein are capable of inhibiting melanin and, hence, capable of brightening the skin tone.

Accordingly, embodiments herein provide a cosmetic method and a dermatological method. The term “cosmetic”, as used herein, refers to its generally accepted meaning. It refers to aspects relating to the appearance of a person or parts thereof such as skin, face, hands, feet, etc. Accordingly, the term “cosmetic method”, as used herein, refers to a method for treating a person or parts thereof such as skin, face, etc, to improve appearance. In an embodiment, the cosmetic method is a non-medical method, preferably involving applying preparations, topically, to skin, face etc. In an embodiment, the cosmetic method is a non-medical method, preferably involving applying preparations, topically, to a person to achieve skin brightening, maintain even skin tone, decrease sebum secretion in skin, reduce inflammation, improve skin moisturization and/or control trans epidermal water loss.

The term “dermatologic” or “dermatological”, as used herein, means that relating to skin. Accordingly, the term “dermatological method”, as used herein, refers to a method for treating a person or parts thereof such as skin, face, etc, to manage or prevent skin conditions, for eg: dermatitis. In an embodiment, the dermatological method is a medical method, preferably involving administering preparations to a person to treat dermatological conditions selected from dermatitis, atopic dermatitis, acne, skin rashes and skin inflammation.

### Marigold Extract

Embodiments herein achieve extracts of marigold comprising one or more flavonoids, also referred to herein as a complex of flavonoids. The flavonoids, according to embodiments herein, are selected from quercetagetin, patuletin, kaempherol, 6-hydroxykaempherol, quercetin, galangin, or combination thereof. In an embodiment, the extract of marigold is a marigold extract comprising quercetagetin, wherein the amount of quercetagetin in the extract is in an amount in the range of 40 wt % to 99 wt %. In an embodiment, the marigold extract comprises quercetagetin in an amount of at least 40 wt %, at least 45 wt %, at least 50 wt %, at least 55 wt %, at least 60 wt %, at least 65 wt %, at least 70 wt %, at least 75 wt %, at least 80 wt %, at least 85 wt %, at least 90 wt %, at least 95 wt %, or at least 99 wt %. In an embodiment, the marigold extract comprises quercetagetin in an amount of at least 40 wt % to 99 wt %.

Based on the amount of quercetagetin present in the marigold extract, the extract is referred to herein as “first extract”, “second extract” and “marigold extract enriched with quercetagetin”. In an embodiment, the marigold extract is obtained by the method as disclosed in various embodiments herein. In an embodiment, the marigold extract comprises quercetagetin in an amount of at least 40 wt % to 60 wt %, also referred to herein as “first extract” or “QG40”. The term “first extract” or “QG40”, as used herein refers to the marigold extract comprising quercetagetin in an amount of at least 40 wt % to 60 wt % in respect of the total weight of the marigold extract. In another embodiment, the marigold extract comprises quercetagetin in an amount of at least 60 wt % to 75 wt %, also referred to herein as “second extract”. The term “second extract”, as used herein refers to the marigold extract comprising quercetagetin in an amount of at least 60 wt % to 75 wt % in respect of the total weight of the marigold extract. In another embodiment, the marigold extract comprises quercetagetin in an amount of at least 90 wt %, also referred to herein as “marigold extract enriched with quercetagetin” or “QG90”. The term “marigold extract enriched with quercetagetin” or “QG90”, as used herein refers to the marigold extract comprising quercetagetin in an amount of at least 90 wt % in respect of the total weight of the marigold extract. The term “marigold extract” broadly refers to all the extracts of marigold of the present disclosure. It is used interchangeably herein with the terms “first extract”, “second extract” or “marigold extract enriched with quercetagetin”. Hence, the term marigold extract refers to QG40 or QG90 or second extract.

In an embodiment, the amount of flavonoids selected from patuletin, kaempherol, 6-hydroxykaempherol, quercetin, galangin, or combination thereof, may vary based on the amount of quercetagetin in the enriched marigold extract.

The amount of flavonoids selected from patuletin, kaempherol, 6-hydroxykaempherol, quercetin, galangin, or combination thereof, may vary from 0 to 10 wt % in respect of eth extract.

In an embodiment, the marigold extract comprises patuletin in an amount ranging from 1 wt % to 7 wt %. In an embodiment, the marigold extract comprises kaempherol compounds an amount ranging from 0 to 5 wt %. In an embodiment, the marigold extract comprises kaempherol compounds selected from 6-hydroxykaempherol, kaempherol, or combination thereof, wherein 6-hydroxykaempherol is present in an amount of 0 to 4 wt %, and kaempherol is present in an amount of 0 to 1 wt %. In an embodiment, the marigold extract comprises galangin in an amount ranging from 0.001 wt % to 0.005 wt %. In an embodiment, the marigold extract comprises quercetin in an amount ranging from 0.001 wt % to 0.005 wt %.

The marigold extract, according to embodiments herein, further comprises one of more phytosterols. The phytosterols are selected from stigmasterol, beta-sitosterol, Beta-sitosterol-Beta-D-glycoside or combination thereof. In an embodiment, the marigold extract comprises phytosterols in an amount ranging from 0 to 2.5 wt %. In an embodiment, the marigold extract comprises stigmasterol in an amount ranging from 0.05 wt % to 1 wt %. In an embodiment, the marigold extract comprises stigmasterol in an amount ranging from 0.05 wt % to 1 wt %. In an embodiment, the marigold extract comprises beta-sitosterol in an amount ranging from 0.01 wt % to 1.5 wt %. In an embodiment, the marigold extract comprises beta-sitosterol-beta-D-glycoside in an amount ranging from 0.05 wt % to 1 wt %.

The marigold extract, according to embodiments herein, further comprises at least one nutrient selected from carbohydrate, protein, sugar, fat, crude fibre, or combination thereof. In an embodiment, the marigold extract comprises at least one nutrient selected from carbohydrate, protein, sugar, fat, crude fibre, or combination thereof. In an embodiment, the marigold extract comprises the nutrient in an amount ranging from 0.1 wt % to 40 wt %. In an embodiment, the marigold extract comprises the nutrient in an amount ranging from at least 3 wt % to 35 wt % or at least 4 wt % to 34 wt %. In an embodiment, the marigold extract comprises the nutrient in an amount of at least 33 wt %, at least 17 wt %, or at least 4 wt %.

In an embodiment, the nutrient is carbohydrate in an amount ranging from 2 wt % to 25 wt %. In an embodiment, the marigold extract comprises carbohydrate in an amount of at least 2.5 wt %, at least 12 wt %, or at least 22 wt %.

In an embodiment, the nutrient is protein in an amount ranging from 0.1 wt % to 6 wt %. In an embodiment, the marigold extract comprises protein in an amount of at least 0.1 wt %, at least 2.1 wt %, or at least 5.1 wt %. In an embodiment, the nutrient is sugar in an amount ranging from 0.1 wt % to 2 wt %. In an embodiment, the marigold extract comprises sugar in an amount of at least 1.2 wt %, at least 1.3 wt %, or at least 1.5 wt %. In an embodiment, the nutrient is fat in an amount ranging from 0.1 wt % to 5 wt %. In an embodiment, the marigold extract comprises fat in an amount of at least 0.65 wt %, at least 1.85 wt %, or at least 4.85 wt %.

In an embodiment, the nutrient is crude fibre in an amount ranging from 0.01 wt % to 1 wt %. In an embodiment, the nutrient is crude fibre in an amount of 0.1 wt %. In an embodiment, the marigold extract further comprises ash content in an amount ranging from 0.5 wt % to 3.5 wt %. In an embodiment, the marigold extract further comprises ash content in an amount of at least 1.5 wt %, or at least 2.5 wt %.

In an embodiment, the marigold extract further comprises ash content in an amount ranging from 0.5 wt % to 3.5 wt %. In an embodiment, the marigold extract further comprises ash content in an amount of at least 1.5 wt %, or at least 2.5 wt %.

In an embodiment, the marigold extract further comprises water content in an amount ranging from 2.5 wt % to 5 wt %, preferably 3 wt % to 4.5 wt %. In an embodiment, the marigold extract further comprises water content in an amount of at least 3.1 wt % or at least 4.1 wt %.

### Composition

Embodiments herein achieve compositions comprising marigold extract, wherein the marigold extract comprises one or more flavonoids. In an embodiment, the composition comprises marigold extract, wherein the marigold extract comprises one or more flavonoids, one or more phytosterols, and one or more nutrient. In an embodiment, the composition comprises marigold extract, wherein the marigold extract comprises one or more flavonoids, one or more phytosterols, and one or more nutrient; and a cosmetically acceptable excipient.

The flavonoids, as described previously herein, are selected from quercetagetin, patuletin, kaempherol, 6-hydroxykaempherol, quercetin, galangin, or combination thereof. In an embodiment, the composition comprises a marigold extract enriched with quercetagetin, wherein the amount of quercetagetin in the extract is in an amount in the range of 40 wt % to 99 wt % in respect of the marigold extract. In an embodiment, the composition comprises a marigold extract, wherein the marigold extract comprises quercetagetin in an amount of at least 40 wt %, at least 45 wt %, at least 50 wt %, at least 55 wt %, at least 60 wt %, at least 65 wt %, at least 70 wt %, at least 75 wt %, at least 80 wt %, at least 85 wt %, at least 90 wt %, at least 95 wt %, or at least 99 wt %, in respect of the extract. In another embodiment, the composition comprises a marigold extract, wherein the marigold extract comprises quercetagetin in an amount of at least 40 wt % to 60 wt % in respect of the extract. Accordingly, in an embodiment, the composition comprises the first extract. In another embodiment, the composition comprises a marigold extract, wherein the marigold extract comprises quercetagetin in an amount of at least 40 wt % to 75 wt %, in respect of the extract. In another embodiment, the composition comprises a marigold extract, wherein the marigold extract comprises quercetagetin in an amount of at least 60 wt % to 75 wt %, in respect of the extract. Accordingly, in an embodiment, the composition comprises the second extract. In another embodiment, the composition comprises a marigold extract, wherein the marigold extract comprises quercetagetin in an amount of at least 90 wt %, in respect of the extract. Accordingly, in an embodiment, the composition comprises the marigold extract enriched with quercetagetin.

In another embodiment, the composition comprises a marigold extract, wherein the marigold extract comprises flavonoids selected from patuletin, kaempherol, 6-hydroxykaempherol, quercetin, galangin, or combination thereof, may vary from 0 to 10 wt % in respect of the extract.

In another embodiment, the composition comprises a marigold extract, wherein the marigold extract comprises patuletin in an amount ranging from 1 wt % to 7 wt % in respect of the extract. In another embodiment, the composition comprises a marigold extract, wherein the marigold extract comprises kaempherol compounds in an amount ranging from 0 to 5 wt % in respect of the extract. In another embodiment, the composition comprises a marigold extract, wherein the marigold extract comprises kaempherol compounds selected from 6-hydroxykaempherol, kaempherol, or combination thereof, wherein 6-hydroxykaempherol is present in an amount of 0 to 4 wt %, and kaempherol is present in an amount of 0 to 1 wt %. In another embodiment, the composition comprises a marigold extract, wherein the marigold extract comprises galangin in an amount ranging from 0.001 wt % to 0.005 wt % in respect of the extract. In another embodiment, the composition comprises a marigold extract, wherein the marigold extract comprises quercetin in an amount ranging from 0.001 wt % to 0.005 wt % in respect of the extract.

The composition comprising the marigold extract, according to embodiments herein, further comprises one of more phytosterols. The phytosterols, as previously described herein, are selected from stigmasterol, beta-sitosterol, or derivatives thereof. The derivative of phytosterol, according to embodiments herein, is Beta-sitosterol-Beta-D-glycoside. In an embodiment, the composition comprises a marigold extract, wherein the marigold extract comprises phytosterols in an amount ranging from 0 to 2.5 wt % in respect of the extract. In an embodiment, the composition comprises a marigold extract, wherein the marigold extract comprises stigmasterol in an amount ranging from 0.05 wt % to 1 wt % in respect of eth extract. In an embodiment, the composition comprises a marigold extract, wherein the marigold extract comprises stigmasterol in an amount ranging from 0.05 wt % to 1 wt % in respect of the extract. In an embodiment, the composition comprises a marigold extract, wherein the marigold extract comprises beta-sitosterol in an amount ranging from 0.01 wt % to 1.5 wt % in respect of the extract. In an embodiment, the composition comprises a marigold extract, wherein the marigold extract comprises beta-sitosterol-beta-D-glycoside in an amount ranging from 0.05 wt % to 1 wt % in respect of the extract.

The composition comprising the marigold extract, according to embodiments herein, further comprises at least one nutrient selected from carbohydrate, protein, sugar, fat, crude fibre, or combination thereof. In an embodiment, the composition comprises a marigold extract, wherein the marigold extract comprises at least one nutrient selected from carbohydrate, protein, sugar, fat, crude fibre, or combination thereof. In an embodiment, the composition comprises a marigold extract, wherein the marigold extract comprises nutrient in an amount ranging from 0.1 wt % to 40 wt %. The amount of nutrient in the extract may be in an amount ranging from at least 3 wt % to 35 wt % or at least 4 wt % to 34 wt %. In an embodiment, the composition comprises a marigold extract, wherein the marigold extract comprises the nutrient in an amount of at least 33 wt %, at least 17 wt %, or at least 4 wt %.

In an embodiment, the composition comprises a marigold extract, wherein the marigold extract comprises carbohydrate in an amount ranging from 2 wt % to 25 wt % in respect of the extract. In an embodiment, the composition comprises a marigold extract, wherein the marigold extract comprises carbohydrate in an amount of at least 2.5 wt %, at least 12 wt %, or at least 22 wt % in respect of the extract.

In an embodiment, the nutrient is protein in an amount ranging from 0.1 wt % to 6 wt %. In an embodiment, the composition comprises a marigold extract, wherein the marigold extract comprises protein in an amount of at least 0.1 wt %, at least 2.1 wt %, or at least 5.1 wt %. In an embodiment, the nutrient is sugar in an amount ranging from 0.1 wt % to 2 wt %. In an embodiment, the composition comprises a marigold extract, wherein the marigold extract comprises sugar in an amount of at least 1.2 wt %, at least 1.3 wt %, or at least 1.5 wt % in respect of the extract. In an embodiment, the nutrient is fat in an amount ranging from 0.1 wt % to 5 wt % in respect of the extract. In an embodiment, the composition comprises a marigold extract, wherein the marigold extract comprises fat in an amount of at least 0.65 wt %, at least 1.85 wt %, or at least 4.85 wt % in respect of the extract.

In an embodiment, the nutrient is crude fibre in an amount ranging from 0.01 wt % to 1 wt % in respect of the extract. In an embodiment, the composition comprises a marigold extract, wherein the marigold extract comprises crude fibre in an amount of 0.1 wt % in respect of the extract. In an embodiment, the composition comprises a marigold extract, wherein the marigold extract comprises ash content in an amount ranging from 0.5 wt % to 3.5 wt % in respect of the extract. In an embodiment, the composition comprises a marigold extract, wherein the marigold extract comprises ash content in an amount of at least 1.5 wt %, or at least 2.5 wt % in respect of the extract.

In an embodiment, the marigold extract comprises ash content in an amount ranging from 0.5 wt % to 3.5 wt % in respect of the extract. In an embodiment, the marigold extract further comprises ash content in an amount of at least 1.5 wt %, or at least 2.5 wt % in respect of the extract. In an embodiment, the marigold extract further comprises water content in an amount ranging from 2.5 wt % to 5 wt %, preferably 3 wt % to 4.5 wt % in respect of the extract. In an embodiment, the marigold extract further comprises water content in an amount of at least 3.1 wt % or at least 4.1 wt % in respect of the extract.

The composition may be formulated in any suitable form. Examples of suitable forms include semi-solids such as creams, gels, foam, and ointment; liquids such as suspensions, emulsions, liniment and lotions; solid such as soaps, patches and powders; and gaseous such as vapors and sprays. A person skilled in the art would be able to combine commonly used cosmetically acceptable excipient in order to achieve the various forms of the composition. The term “cosmetically acceptable carriers” refers substances that are suitable for contact with keratin tissue, for eg: skin, of a subject. It refers to substances that are safe for application on keratin tissue of a subject without causing any toxicity, allergy, and the like in the subject. Examples of cosmetically acceptable excipient for solid forms include, but is not limited to, starch, maltodextrin and cyclodextrin; semi-solids forms include, but is not limited to polysorbate, glyceryl stearate and lecithin; liquid forms include, but is not limited to Glycerin, Aqua, ethanol and DMSO (Dimethyl sulfoxide).

The amount of marigold in the composition may vary depending on application, form, etc, of the composition. In an embodiment, the composition comprises the marigold extract in an amount ranging from at least 0.01 wt % to 5 wt %, at least 0.05 wt % to 4 wt %, at least 0.1 wt % to 3 wt %, or at least 0.5 wt % to 2 wt %, in respect of the composition. In an embodiment, the composition comprises the marigold extract in an amount of 0.5 wt %. In another embodiment, the composition comprises the marigold extract in an amount of 1 wt %. In an embodiment, the composition comprises the marigold extract in an amount of 1.5 wt %.

In another embodiment, the composition is a cream comprising 0.01 wt % to 5 wt %, at least 0.05 wt % to 4 wt %, at least 0.1 wt % to 3 wt %, or at least 0.5 wt % to 2 wt % of the marigold extract, in respect of the composition. In an embodiment, the composition is a cream comprising the marigold extract in an amount of 0.5 wt %. In another embodiment, the composition is a cream comprising the marigold extract in an amount of 1 wt %. In an embodiment, the composition is a cream comprising the marigold extract in an amount of 1.5 wt %.

In another embodiment, the composition is a cream comprising at least 0.01 wt % to 5 wt %, at least 0.05 wt % to 4 wt %, at least 0.1 wt % to 3 wt %, or at least 0.5 wt % to 2 wt % of the marigold extract, wherein the marigold extract comprises quercetagetin is present in an amount in the range of 40 wt % to 99 wt % in respect of the marigold extract. The amount of quercetagetin in the composition, wherein the composition is a cream, may be an amount of at least 40 wt %, at least 45 wt %, at least 50 wt %, at least 55 wt %, at least 60 wt %, at least 65 wt %, at least 70 wt %, at least 75 wt %, at least 80 wt %, at least 85 wt %, at least 90 wt %, at least 95 wt %, or at least 99 wt %, in respect of the extract. In another embodiment, the composition is a cream comprising the first extract. In another embodiment, the composition is a cream comprising the second extract. In another embodiment, the composition is a cream comprising the marigold extract enriched with quercetagetin, wherein quercetagetin is present in an amount in the range of at least 40 wt % to 99 wt %, or at least 60 wt % to 75 wt %, or at least 80 wt % to 90 wt %.

The cream, according to embodiments herein, may further include one or more cosmetically acceptable excipient. In an embodiment, the cream includes Acrylates/C10-30 Alkyl Acrylate Crosspolymer, Triethanolamine, Glycerin, Glyceryl Monostearate, CetoStearyl Alcohol, Glycol Stearate & PEG (100) Stearate, Prunus Amygdalus Dulcis (Sweet Almond) Oil, Argania Spinosa Kernel Seed Oil, Caprylic Capric Triglyceride, Isopropyl Myristate, Glyceryl Caprylate, Glyceryl Undecylenate, Tocopherol, Helianthus Annuus Seed Oil, Phenoxyethanol, Propylene Glycol, or combinations thereof. The amount of excipients may be in the range of 90 to 99.9 wt %. In an embodiment, the amount of excipient is in an amount of 99.0 wt %.

In an exemplary embodiment, the composition is a cream comprising the disclosed marigold extract in an amount in the range of 0.1 to 5 wt %; almond oil in an amount in the range of 1 to 5 wt %; emulsifiers in an amount in the range of 10 to 90 wt %; demineralised water in the range of 10-90%; surfactants in an amount in the range of 1-10%; and solvents in an amount in the range of 2-5%.

In another embodiment, the composition is a gel comprising at least 0.01 wt % to 5 wt %, at least 0.05 wt % to 4 wt %, at least 0.1 wt % to 3 wt %, or at least 0.5 wt % to 2 wt % of the marigold extract, in respect of the composition. In an embodiment, the composition is a gel comprising the marigold extract in an amount of 0.5 wt %. In another embodiment, the composition is a gel comprising the marigold extract in an amount of 1 wt %. In an embodiment, the composition is a gel comprising the marigold extract in an amount of 1.5 wt %.

In another embodiment, the composition is a gel comprising 0.01 wt % to 5 wt %, at least 0.05 wt % to 4 wt %, at least 0.1 wt % to 3 wt %, or at least 0.5 wt % to 2 wt % of the marigold extract, wherein the marigold extract comprises quercetagetin in an amount in the range of at least 40 wt % to 99 wt % in respect of the marigold extract. The amount of quercetagetin in the composition, wherein the composition is a gel, may be an amount of at least 40 wt %, at least 45 wt %, at least 50 wt %, at least 55 wt %, at least 60 wt %, at least 65 wt %, at least 70 wt %, at least 75 wt %, at least 80 wt %, at least 85 wt %, at least 90 wt %, at least 95 wt %, or at least 99 wt %, in respect of the extract. In another embodiment, the composition is a gel comprising the first extract. In another embodiment, the composition is a gel comprising the second extract. In another embodiment, the composition is a gel comprising the marigold extract enriched with quercetagetin, wherein quercetagetin is present in an amount in the range of at least 40 wt % to 99 wt %, or at least 60 wt % to 75 wt %, or at least 80 wt % to 90 wt % in respect of weight of the marigold extract.

The gel, according to embodiments herein, may further include one or more cosmetically acceptable excipient selected from gums, emulsifiers, copolymers. In an embodiment, the gel includes Aqua, gums, emulsifiers, copolymers. The amount of excipients may be in the range of 80 to 99 wt %. In an embodiment, the amount of excipient is in the range of 90-99% wt in respect of the total weight of the gel.

The composition, according to embodiments herein, further comprises at least one cosmetically acceptable excipient. The cosmetically acceptable excipient, according to embodiments herein, is selected from emulsifiers, antioxidants, diluents, emollients, antimicrobial agents, pH modulating agents, preservatives, or combinations thereof.

In an embodiment, the emulsifier is selected from sodium lauryl sulphate, benzalkonium chloride and behentrimonium methosulphate, cetostearyl alcohol, cetostearyl alcohol, cetostearyl glucoside, glyceryl stearate, cetostearyl alcohol and sodium stearoyl lactylate, stearic acid, glyceryl stearate, peg-100 stearate, glyceryl stearate, or combination thereof.

In an embodiment, the antioxidant is selected from vitamin C, niacinamide, resveratrol, vitamin E, retinol, Coenzyme Q10, polyphenols, or combination thereof.

In an embodiment, the diluent is selected from water, glycerin, glycerol and aloe vera gel, or combination thereof.

In an embodiment, the emollient is selected from beeswax, coconut oil, almond oil, argan oil, caprylic capric triglyceride, shea butter, cocoa butter, lanolin, tocopherols, avocado oil, propylene glycol, isopropyl myristate, castor oil, or combination thereof.

In an embodiment, the antimicrobial agent is selected from glyceryl caprylate, glyceryl undecylenate, or combination thereof.

In an embodiment, the pH modulating agent is selected from triethanolamine, citric acid, lactic acid, sodium lactate, or combination thereof.

In an embodiment, the preservative is selected from ethylparaben, methylparaben, potassium diazolidinyl urea, benzyl alcohol, phenoxyethanol, sodium benzoate, or combination thereof.

In an embodiment, the composition is a cream comprising the marigold extract and the cosmetically acceptable carrier selected from emulsifiers, antioxidants, diluents, emollients, antimicrobial agents, pH modulating agents, preservatives, or combinations thereof.

In an embodiment, the composition is a gel comprising the marigold extract and the cosmetically acceptable carrier selected from emulsifiers, antioxidants, diluents, emollients, antimicrobial agents, pH modulating agents, preservatives, or combinations thereof.

### Method

Embodiments herein provide a method for preparing the disclosed marigold extract. The method, according to embodiments herein, is capable of preparing a marigold extract having quercetagetin. In an embodiment, the method comprises (a) subjecting marigold biomass to extraction using an alcohol selected from ethanol, methanol, or combination thereof, to obtain a miscella fraction; and (b) evaporating the miscella fraction to obtain the marigold extract. In an embodiment, the method comprises (a) subjecting marigold biomass to extraction using an alcohol selected from ethanol, methanol, or combination thereof, to obtain a miscella fraction; (b) evaporating the miscella fraction to obtain a first extract; (c) treating the first extract with a solvent comprising n-hexane to obtain a residue; and (d) drying the residue to obtain the marigold extract.

The method for preparing the marigold extract, according to embodiments herein, includes the use of marigold biomass as a starting material. The term “marigold biomass”, as used herein, refers to organic material derived from marigold, preferably marigold flower. In an embodiment, the marigold biomass comprises silaged and shredded marigold flowers. Preferably, the silaged and shredded flowers are further compressed to remove moisture such that the water content of the marigold biomass is in the range of 60-65 wt %. Typically, silaging is performed by subjecting plant material to anaerobic bacterial fermentation. It is fermented mass resulting from the storage of plant material under anaerobic conditions in structures called silos. In an embodiment, the marigold flowers are silaged under anaerobic condition for 15 to 90 days. Shredding and compressing of the marigold flowers may be performed by methods generally known in the art using for eg: mechanical shredder, compressor, etc.

Accordingly, the marigold biomass, in an embodiment, is obtained by silaging, shredding and compressing marigold flowers to remove moisture. In another embodiment, the marigold biomass is obtained by silaging, shredding and compressing marigold flowers; and, optionally, dehydrating by treating with dimethyl ether. In an embodiment, the marigold flowers are silage, shredded, compressed and treated with dimethyl ether to dehydrate the flowers and obtain marigold biomass. Accordingly, in an embodiment, the method for preparing the disclosed marigold extract comprises silaging, shredding, and compressing of marigold flowers; and optionally, treating with dimethyl ether. In an embodiment, the water content in marigold biomass is in the range of 60-65 wt %, in respect of the total weight of the biomass. In an embodiment, the dimethyl ether is used as an extraction solvent to dehydrate the flowers, wherein the dimethyl ether is passed through an extraction vessel comprising the silage, shredded and compressed marigold flowers. The marigold biomass is obtained by separation using centrifugation.

The step of subjecting the marigold biomass to extraction, in the method according to embodiments herein, comprises extracting the marigold biomass using alcohol as extractant, preferably selected from ethanol, methanol, or combination thereof. The term “extraction” or “extracting”, as used herein, refers to transference of substances from one phase to another. The phase may be solid or liquid, accordingly, the extraction may be liquid-liquid extraction or solid-liquid extraction. The alcohol, in an embodiment herein, is used to extract quercetagetin from the marigold biomass. The extraction process may be performed one or more times. In an embodiment, the extraction using alcohol is performed one time. The extraction may be performed in a solvent extractor. The extraction process may be performed for a period in the range of 2 to 4 hrs and may be repeated one or more times.

In another embodiment, the extraction using alcohol is repeated two to five times to obtain multiple fractions which can then be combined. The extraction using alcohol, in the method according to embodiments herein, achieves a miscella fraction. The term “miscella fraction”, as used herein, is a fraction of oil or solvent mixture that leaves a solvent extractor. In an embodiment, the miscella fraction is the alcohol fraction obtained after the extraction is complete. In an embodiment, the miscella fraction is an ethanol fraction comprising quercetagetin obtained after the extraction of the marigold is complete. In an embodiment, the miscella fraction is a methanol fraction comprising quercetagetin obtained after the extraction of the marigold.

In an embodiment, the ratio of the marigold biomass and alcohol is in a weight ratio in the range of 1:3 to 1:5. The miscella fraction is then subjected to evaporation to obtain a first extract. Accordingly, the method, as disclosed herein, comprises evaporating the miscella fraction to obtain a first extract. The evaporation, as disclosed herein, may be performed using methods generally known in the art. In an embodiment, evaporation is performed using a vacuum oven, preferably dry air circulation; at a temperature preferably 60-80 degrees Celsius, for period in the range of 60 to 80 minutes, preferably 60 minutes.

In an embodiment, the first extract is a marigold extract, wherein the quercetagetin is in the range of at least 40-60 wt % in respect of the total weight of the extract. In an embodiment, the first extract is a green waxy product.

Embodiments herein comprise the step of treating the first extract with a solvent comprising n-hexane to obtain a residue. This step is performed to preferably separate the lipophilic compounds, wherein said lipophilic compound is lutein. The first extract, according to embodiments, is treated by extracting the first extract with the solvent comprising n-hexane. In an embodiment, the solvent is n-hexane. In another embodiment, the solvent comprises n-hexane and a solvent selected from acetone, ethyl acetate, or combination thereof.

In an embodiment, the ratio of first extract and solvent is in a weight ratio in the range of 1:3 to 1:5. In an embodiment, the ratio of first extract and solvent used in the extraction is 1:3, 1:4 or 1:5.

The residue obtained after the treating of the first extract with solvent is then dried to achieve further enrichment of quercetagetin. In an embodiment, the method comprises drying the residue to obtain marigold extract comprising quercetagetin in the range of at least 60-75 wt %.

Drying may be performed by methods generally known in the art. In an embodiment, the drying is performed using a vacuum oven, preferably dry air circulation; at a temperature 60-80, preferably 60 degrees Celsius, for period in the range of 60 to 80 minutes, preferably 60 minutes.

The residue, according to embodiments, may further be enriched to obtain a marigold extract having more than 75 wt % or at least 90 wt % of quercetagetin. The residue may be dried which may further be enriched. Accordingly, in an embodiment, the method comprises (a) subjecting marigold biomass to extraction using an alcohol selected from ethanol, methanol, or combination thereof, to obtain a miscella fraction; (b) evaporating the miscella fraction to obtain a first extract; (c) treating the first extract with a solvent comprising n-hexane to obtain a residue; (d) drying the residue to obtain a second extract; and (e) enriching by treating the second extract with ethanol and activated carbon, to obtain the marigold extract.

The step of enrichment by treating the second extract with ethanol and activated carbon, in the method as disclosed herein, comprises mixing the second extract with ethanol at a temperature in the range of 55-70° C., preferably 60-65° C.; adding the activated carbon; stirring for a period of 20 to 24 hrs at 20 to 27 degree temperature; and filtering to remove the activated carbon and distilling to remove the ethanol. Filtration and distillation may be performed using methods known in the art. Filtration is performed to remove the activated carbon by using a suitable filtration membrane.

In the enrichment step, the ratio of second extract and ethanol is in a weight ratio in the range of 1:30 to 1:50. In the enrichment step, the ratio of second extract and ethanol is in a weight ratio in the range of 1:40.

In an example, the second extract is a pale green extract comprising 75 wt % of quercetagetin which is dissolved in ethanol and stirred at 60-65 degrees Celsius. Further, activated carbon is added to obtain a mixture which is stirred for about 20-24 hours. The activated carbon is then removed by filtration, and preferably the carbon is washed using ethanol, to obtain a filtrate. Once the activated carbon is removed, water is added to the filtrate, and the ethanol is distilled out to obtain the marigold extract having more than 75 wt % of quercetagetin. In an embodiment, the marigold extract enriched with quercetagetin, comprises quercetagetin in an amount of in the range of at least 75 to 90 wt %, or 75 to 99 wt % to the total weight of the marigold extract.

In an embodiment, the marigold extract enriched with quercetagetin, comprises quercetagetin in an amount of at least 90 wt %.

The marigold extract enriched with quercetagetin, comprises no or non-detectable amounts of lipophilic compounds, wherein said lipophilic compound is lutein.

The method, according to embodiments herein, may further include repetition of one or more steps to achieve higher or complete extraction. For example, the marigold biomass, first extract, second extract, etc. may be subjected to one or more rounds of extraction, treatment, enriching, etc. to achieve higher or complete extraction. In an example, the steps (a) and (b), of the method as disclosed herein, may optionally be repeated, wherein said extracting of marigold biomass using alcohol (i.e. step (a)) and said evaporating the miscella fraction (i.e. step (b)) may be repeated to obtain multiple fractions of the first extract which may then be combined and subjected to the remaining method steps to obtain the second extract comprising quercetagetin in the range of at least 60-75 wt % in respect of the second extract extract, and marigold extract enriched with quercetagetin comprising quercetagetin in an amount of at least 90 wt %. Alternatively, step (a) may optionally be repeated, wherein said extracting of marigold biomass using alcohol (i.e. step (a)) may be repeated to obtain multiple fractions which may then be combined and evaporated to obtain the first extract.

Similarly, step (c) and (d), of the method as disclosed herein, may optionally be repeated, wherein said treating the first extract using a solvent (i.e. step (c)) and drying (i.e. step (d)) may be repeated to obtain multiple fractions which may then be combined and subjected to the remaining method steps to obtain the marigold extract enriched with quercetagetin comprising quercetagetin in an amount of at least 90 wt %. Alternatively, the step (c) may optionally be repeated, wherein said treating the first extract using a solvent (i.e. step (c)) may be repeated to obtain the multiple fractions which may then be combined and dried to obtain the second extract. The second extract thus obtained may then be subjected to the remaining method steps to obtain the marigold extract enriched with quercetagetin comprising quercetagetin in an amount of at least 90 wt %.

Similarly, step (e) and (f), of the method as disclosed herein, may optionally be repeated, wherein said enriching by treating the second extract with ethanol and activated carbon (i.e. step (e)) and removing the activated carbon and ethanol (i.e. step (f)) may be repeated to obtain multiple fractions which may then be combined to obtain the marigold extract enriched with quercetagetin comprising quercetagetin in an amount of at least 90 wt %. Alternatively, the step (e) may optionally be repeated, enriching by treating the second extract with ethanol and activated carbon (i.e. step (e)) may be repeated to obtain the multiple fractions which may then be combined and subjected to step (f) to obtain the marigold extract enriched with quercetagetin comprising quercetagetin in an amount of at least 90 wt %.

In an embodiment of the present disclosure, there is provided a method for preparing a marigold extract enriched with quercetagetin, wherein step (a), or step (a) and step (b) are repeated to obtain multiple fractions which are combined. In another embodiment of the present disclosure, there is provided a method for preparing a marigold extract enriched with quercetagetin, wherein step (c), or step (c) and step (d) are repeated to obtain multiple fractions which are combined. In another embodiment, of the present disclosure, there is provided a method for preparing a marigold extract enriched with quercetagetin, wherein step (e), or step (e) and step (f) are repeated to obtain multiple fractions which are combined.

In an embodiment, the method for preparing a marigold extract enriched with quercetagetin comprises (a) extracting marigold biomass using an alcohol selected from ethanol and methanol, optionally repeating said extracting, to obtain a miscella fraction, wherein the ratio of biomass extract and alcohol is in the range of 1:3 to 1:5; (b) evaporating the miscella fraction to obtain a first extract comprising quercetagetin in the range of at least 40-60 wt % in respect of the first extract; (c) treating the first extract with a solvent comprising n-hexane, optionally repeating said treating, to obtain a residue, wherein the ratio of first extract and solvent is in the range of 1:3 to 1:5; (d) drying the residue to remove the solvent and obtain a second extract comprising quercetagetin in the range of at least 60-75 wt % in respect of the second extract; (e) enriching by treating the second extract with ethanol and activated carbon, optionally repeating said enriching; (f) removing the activated carbon, preferably by filtering, and ethanol, preferably by distillation, to obtain the quercetagetin enriched marigold extract comprising quercetagetin in an amount of at least 90 wt % in respect of the quercetagetin enriched marigold extract, wherein the ratio of second extract and ethanol is in the range of 1:30 to 1:50.

In an aspect of the present disclosure, there is provided a method for preparing a marigold extract enriched with quercetagetin, said method comprising: (a) extracting marigold biomass using an alcohol selected from ethanol and methanol, to obtain a miscella fraction, wherein the ratio of biomass extract and alcohol is in the range of 1:3 to 1:5; (b) evaporating the miscella fraction to obtain a first extract comprising quercetagetin in the range of at least 40-55 wt % in respect of the first extract; (c) optionally repeating step (a) and (b); (d) treating the first extract with a solvent comprising n-hexane to obtain a residue, wherein the ratio of first extract and solvent is in the range of 1:3 to 1:5; (e) drying the residue to remove the solvent and obtain a second extract comprising quercetagetin in the range of at least 60-75 wt % in respect of the second extract; (f) optionally repeating step (d) and (e); (g) enriching by treating the second extract with ethanol and activated carbon, wherein the ratio of second extract and ethanol is in the range of 1:30 to 1:50; (h) removing the activated carbon, preferably by filtering, and ethanol, preferably by distillation, to obtain the marigold extract enriched with quercetagetin comprising quercetagetin in an amount of at least 90 wt %; and (i) optionally repeating step (g) and (h) to obtain the marigold extract.

Embodiments herein further comprises a method for preparing the disclosed composition. In an embodiment, the method comprises blending the marigold extract; and the cosmetically acceptable carrier to obtain the composition. In an embodiment, blending includes preparing an aqueous phase by mixing water-soluble ingredients of the composition; preparing a non-aqueous phase by mixing water insoluble ingredients of the composition; emulsifying the marigold extract using a solvent, preferably propylene glycol; and mixing the emulsified marigold extract, the non-aqueous phase and the aqueous phase, preferably using a blender, to obtain a homogenous blend. Water soluble and water insoluble ingredients of the composition may be identified from the various ingredients described herein and blended separately, such identification and blending would be apparent to a person skilled in the art from the foregoing description. Cream and gel compositions may be prepared using the method described herein above.

Embodiments herein further provide a cosmetic method. In an embodiment, the cosmetic method comprises applying the composition on a target site on the skin to elicit skin brightening activity, decrease sebum secretion, reduce inflammation, improve skin moisturization and control trans epidermal water loss at the target site.

Embodiments herein further provide a dermatalogical method for treatment of skin. In an embodiment, the dermatalogical method comprises administering, topically, the composition on a target site on the skin to elicit 5α-reductase inhibition, anti-inflammatory activity, anti-acne property and/or skin brightening activity at the target site.

Application or administration of the composition or extract is preferably topical on skin epidermis using a spatula, brush, by hand, etc. Application or administration may vary depending on the subject. In an example, the composition is applied once every day. In another example, the composition is applied twice every day. In yet another example, the composition is applied one a week.

The composition and the marigold extract, according to embodiments herein elicit anti-acne and anti-sebum activity while inducing anti-inflammatory activity at the target site. The term “target site”, as used herein, refers to a location on the skin epidermis of a subject. The term “subject”, as used herein, refers to mammals, preferably humans.

### EXAMPLES

The disclosure will now be illustrated with working examples, which is intended to illustrate the working of disclosure and not intended to take restrictively to imply any limitations on the scope of the present disclosure. Unless defined otherwise, all technical and scientific terms used herein have the same meaning as commonly understood to one of ordinary skill in the art to which this disclosure belongs. Although methods and materials similar or equivalent to those described herein can be used in the practice of the disclosed methods and compositions, the exemplary methods, devices, and materials are described herein. It is to be understood that this disclosure is not limited to particular methods, and experimental conditions described, as such methods and conditions may apply.

### Materials and Methods

### Materials

The procurement details of the components of the disclosed composition are listed in the table below.

Marigold flowers were procured, ethanol was procured from Godavari refineries India, dimethyl ether was procured from Azko Nobel SV, n-hexane was procured from BPCL India, activated carbon was procured from Kalpaka Chemicals India.

### Example 1: Preparation of the Marigold Extract Enriched with Quercetagetin (QG), (Also Referred to Herein as 40 wt % QG or QG40)

1000 kg of marigold flowers were collected and silaged anaerobically for 15 days. The silaged flowers were compressed to remove water content. The water content in the compressed wet biomass is about 60 wt %. Further the wet biomass (200 Kg) washed with 600 Liters of about 90% ethanol (washing was repeated 3 times) to obtain ethanol fraction (also referred to herein as miscella fraction) comprising quercetagetin. The ethanol fractions obtained from repeated washing was pooled and filtered through filter press and the solvent evaporated to get marigold extract which is a waxy solid (14 Kg) containing >40% QG (also referred to herein as “First extract” or “QG40”). Table 1 illustrates the components of the first extract, depicted as QG40, wherein the amount of QG is about 40 to 60 wt %, i.e. 50 wt %.

TABLE 1

| Example 1-Marigold extract | QG40 (Amount in wt %) |
|---|---|
| Quercetagetin % | 50 |
| Beta- sitosterol % | 0.5 |
| Stigmasterol % | 0.25 |
| Galangin % | 0.003 |
| Kaempferol % | 0.005 |
| 6-Hydroxykaempherol % | 3 |
| Quercetin % | 0.5 |
| Patuletin % | 5.5 |
| Beta- sitosterol - Beta -D- | 0.25 |
| glycoside | |
| Carbohydrate % | 22.1 |
| Protein % | 5.1 |
| Sugar % | 1.23 |
| Fat % | 4.85 |
| Ash % | 2.5 |
| Crude Fibre % | 0.112 |
| Moisture % | 4.1 |

### Example 2: Preparation of the Marigold Extract Enriched with Quercetagetin (QG), (Also Referred to Herein as 70 wt % QG or QG70)

14 Kg of the first extract was taken in a solvent extractor. 42 liters of n-hexane was added and the reaction mixture stirred for 1-2 hours. The reaction mixture was then filtered and the residue collected. This n-hexane washing of the collected residue was repeated two times. The residue after filtration contains quercetagetin. The residue is dried to yield pale green marigold extract (8 Kg) having >70% QG (also referred to herein as “Second extract” or “QG70”). Table 2 illustrates the components of the first extract, depicted as QG70, wherein the amount of QG is about 60 to 75 wt %, i.e. 72.4 wt %.

TABLE 2

| Example 2-Marigold extract | QG70 (Amount in wt %) |
|---|---|
| Quercetagetin % | 72.4 |
| Beta- sitosterol % | 0.2 |
| Stigmasterol % | 0.1 |
| Galangin % | ND |
| Kaempferol % | ND |
| 6-Hydroxykaempherol % | 1.5 |
| Quercetin % | 0.5 |
| Patuletin % | 4 |
| Beta- sitosterol - Beta -D- | 0.05 |
| glycoside | |
| Carbohydrate % | 12 |
| Protein % | 2.1 |
| Sugar % | 1.2 |
| Fat % | 1.75 |
| Ash % | 1. |
| Crude Fibre % | 0.1 |
| Moisture % | 3.1 |

### Example 3: Preparation of the Marigold Extract Enriched with Quercetagetin (QG), (Also Referred to Herein as 90 wt % QG or QG90)

8 Kg pale green second extract was dissolved in 266 liter of ethanol by stirring at 60-65 deg C. The concentration of second extract in ethanol was about 3 wt % (8 kg in 266 L ethanol). 8 Kg of activated carbon was then added and the obtained reaction mixture stirred for 24 hrs at room temp. After the completion of 24 hours, the reaction mixture was filtered through a filter press (using filter having poresize of 100 microns, procured by Subodh Engineering Pvt Ltd). The carbon residue collected after filtration was washed using 20 liters of ethanol. The filtrate fractions were combined and concentrated to around ⅓rd of its volume, about 75 liters. 150 liter of water was added to the concentrated fraction. Ethanol was distilled out and the concentrate filtered. The residue obtained after filtration was washed with water to obtain a wet residue. This wet residue is dried to obtain a dry residue (4 Kg) which is a yellow powder having QG>90% (also referred to herein as “marigold extract enriched with quercetagetin” or “QG90”). The moisture or water content of the dry residue is <5 wt %. Table 3 illustrates the components of the first extract, depicted as QG90, wherein the amount of QG is about 75 to 99 wt %, i.e. 90.8 wt %, wherein ND stands for “Not detected”.

TABLE 3

| Example 3-Marigold extract | QG 90 (Amount in wt %) |
|---|---|
| Quercetagetin % | 90.8 |
| Beta- sitosterol % | ND |
| Stigmasterol % | ND |
| Galangin % | ND |
| Kaempferol % | ND |
| 6-Hydroxykaempherol % | ND |
| Quercetin % | ND |
| Patuletin % | 1 |
| Beta- sitosterol - Beta -D-glycoside | ND |
| Carbohydrate % | 2 |
| Protein % | 0.1 |
| Sugar % | 1.25 |
| Fat % | 0.65 |
| Ash % | 1. |
| Crude Fibre % | 0.1 |
| Moisture % | 3.1 |

### Example 4: Preparation of the Cream Composition Comprising the Marigold Extract Enriched with Quercetagetin (QG), (Also Referred to Herein as 90 Wt % QG or QG90)

0.5% marigold extract (QG90, QG70 or QG40, obtained from Examples 1-3) was emulsified in 3 ml of propylene glycol under heating at 60 degree Centigrade, to obtain Phase D. Table 4 provides the ingredients to obtain Phase A, B, C and D. The ingredients, depicted in Table 4, in respective quantities, were blended thoroughly using a blender to obtain Phase A and B, separately. After confirming there was no phase separation, Phase A and B were mixed thoroughly with phase C and D to form a uniform cream. Table 4 depicts an exemplary cream composition, according to embodiments herein. Similarly cream compositions having 1 wt % and 0.3 wt % of marigold extract (QG90, QG70 or QG40) were also prepared.

TABLE 4
Cream composition

| Sr | | | | |
|---|---|---|---|---|
| No. | Phase | Ingredient | INCI | Wt % |
| 1 | A | DM Water | Demineralised Water | 72.25 |
| 2 | | Cosmer 940 | Acrylates/C10-30 Alkyl Acrylate | 0.2 |
| | | | Crosspolymer | |
| 3 | | TEA | Triethanolamine | 0.25 |
| 4 | | Glycerin | Glycerin | 2 |
| 5 | B | GMS | Glyceryl Monostearate | 2 |
| 6 | | Cetostearyl Alcohol | CetoStearyl Alcohol | 4 |
| 7 | | Coscare 2067 | Glycol Stearate & PEG (100) | 3 |
| | | | Stearate | |
| 8 | | Sweet Almond Oil | Prunus Amygdalus Dulcis (Sweet | 1 |
| | | | Almond) Oil | |
| 9 | | Organic Moroccan | Argania Spinosa Kernel Seed Oil | 2 |
| | | Argan Oil | | |
| 10 | | CCTG | Caprylic Capric Triglyceride | 3 |
| 11 | | IPM | Isopropyl Myristate | 4 |
| 12 | | Cosphaderm GeMix | Glyceryl Caprylate, Glyceryl | 1 |
| | | | Undecylenate | |
| 13 | | Cosphaderm T-70 | Tocopherol, Helianthus Annuus Seed | 0.1 |
| | | NON GMO ECO | Oil | |
| 14 | C | Phenoxyethanol | Phenoxyethanol | 1.2 |
| 15 | D | Propylene Glycol | Propylene Glycol | 3 |
| 16 | | QG-90 | Marigold extract | 0.5 |
| | | | Total | 100 |

### Example 5: Preparation of the Gel Composition Comprising the Marigold Extract Enriched with Quercetagetin (QG), (Also Referred to Herein as 90 wt % QG or QG90)

0.5 wt % marigold extract (QG90, QG70 or QG40, obtained from Examples 1-3) was emulsified in 3 ml of propylene glycol under heating at 60 degree Centigrade. This constitutes Phase D. Table 5 depicts the ingredients to obtain Phase A, B, C and D. The ingredients, depicted in Table 5, in respective quantities, were blended thoroughly using a blender to obtain Phase A and B, separately. The blended components, after confirming there is no phase separation, were mixed with phase C and D to form a uniform gel. Table 5 depicts an exemplary gel based face gel composition, according to embodiments herein. Similarly gel based face gel compositions having 1 wt % and 0.3 wt % of marigold extract (QG90, QG70 or QG40) were also prepared.

TABLE 5
Face Gel composition with quercetagetin 90 wt %

| Phase | Sr No. | Ingredient | INCI | Wt % |
|---|---|---|---|---|
| A | 1 | DM Water | Demineralised Water | 92.5 |
| | 2 | Cosphaderm X 64 | Xanthan Gum | 0.1 |
| B | 3 | Propylene Glycol | Propylene Glycol | 3 |
| | 4 | Glycerin | Glycerin | 3 |
| | 5 | Reepol 980 | C10-C30 Alkyl | 0.45 |
| | | | Acrylate Copolymer | |
| C | 6 | TEA | Triethanolamine | 0.45 |
| | 7 | QG-90 | Marigold extract | 0.5 |
| D | 8 | Phenoxyethanol | Phenoxyethanol | 1 |
| | | | Total | 100 |

The composition prepared using QG40 and QG90 extracts; and quercetin were evaluated for its antioxidant activity, Oxygen Radical Absorbance Capacity, Cellular ROS Assay, Cellular Anti-inflammatory Assay, Cellular Melanin Inhibition Assay, PIPT & HRIPT Study and Skin Brightening/Even skin tone clinical Study. The detailed protocol of each of these studies conducted along with the results is discussed below. Quercetin dihydrate having 95.6% purity was used for comparative evaluation.

### In-Vitro Studies

### DPPH Antioxidant Assay:

DPPH assay is considered a valid accurate and easy method to evaluate radical scavenging activity of antioxidants. DPPH [1,1-diphenyl-2-picryl hydrazyl] is a stable free radical with purple color. Antioxidants reduce DPPH to 1,1-diphenyl-2-picryl hydrazine, a color less compound which is measured at an absorbance of 590 nm. This is an accepted mechanism for screening the antioxidant activity of plant extracts in a concentration dependent manner. Accordingly, the DPPH Antioxidant Assay was conducted as per known protocols for the compositions comprising QG40, and compositions comprising QG90, and Quercetin.

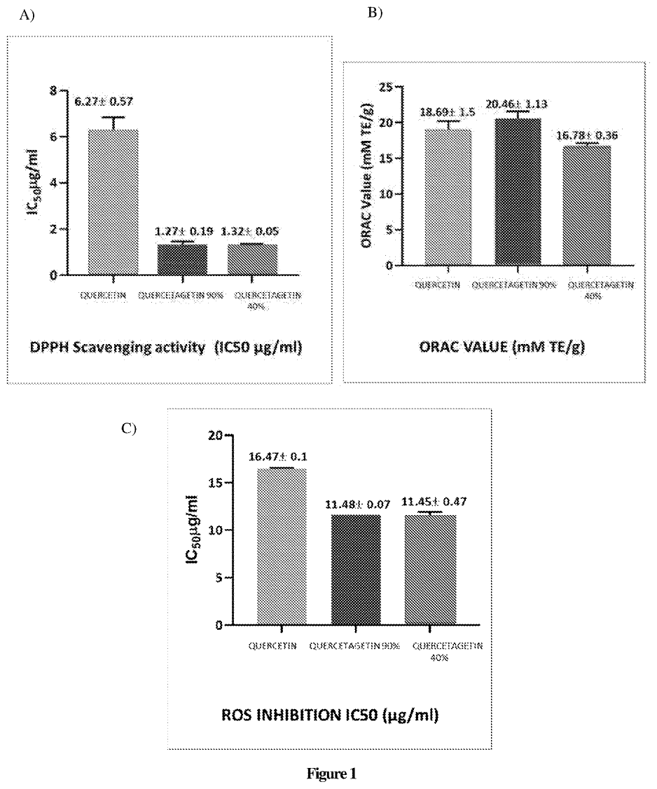

DPPH Scavenging activity (IC50 μg/ml) when evaluated composition with QG40 extract; the composition with QG90 extract and keeping the composition with quercetin as standard; the activity was found as 1.27 1.32; and 6.27 μg/ml respectively (FIG. 1a). For DPPH Scavenging activity, lower the IC50 values, the better the composition.

### Oxygen Radical Absorbance Capacity (ORAC) Assays:

The Oxygen Radical Absorbance Capacity (ORAC) assay is a method that measures the antioxidant capacity of a substance. The ORAC assay measures a fluorescent signal from a probe that is quenched in the presence of Reactive Oxygen Species (ROS). Protocol described in Boxin Ou, et al. Determination of Total Antioxidant Capacity by Oxygen Radical Absorbance Capacity (ORAC) Using Fluorescein as the Fluorescence Probe: First Action 2012.23, Journal of AOAC INTERNATIONAL, Pages 1372-1376, https://doi.org/10.5740/jaoacint.13-175 may be used. In accordance with this protocol, ORAC assay was evaluated (using known protocols) for the composition comprising QG40 extract; the composition comprising QG90 extract and compared the same with composition comprising quercetin. However, ORAC (m1\4 TE/g) values, were found to be 16.78; 20.46 and 18.69 for composition with QG40 extract; the composition with QG90 extract and composition with quercetin, respectively (FIG. 1b). Higher the ORAC value, means better performance by the composition.

### Cellular ROS Assay

The Cellular ROS Assay is a cell-based assay designed for measuring Reactive Oxygen Species activity within a cell using the cell-permeable fluorogenic probe 2′. 7′-Di chloro dihydro fluorescein diacetate (DCF-DA). Keratinocyte (HACAT) cells are pre-treated with various concentrations of test samples and the oxidative stress is induced post-test sample treatment. ROS induces the modification of carboxy-H2DCFDA that fluoresces green as detected by flow cytometry. ROS inhibition was evaluated for the composition with QG40 extract; the composition with QG90 extract and composition with quercetin.

The ROS inhibition IC50 obtained were 11.45; 11.48 and 16.47 μg/ml respectively for quercetagetin 40%; quercetagetin 90% and quercetin (FIG. 1c). Cellular ROS Assay confirms that both composition with QG40 extract; and the composition with QG90 extract are more potent than quercetin. In cellular ROS assay, a lower IC50 is better and desirable.

### Cellular Anti-Inflammatory Assay

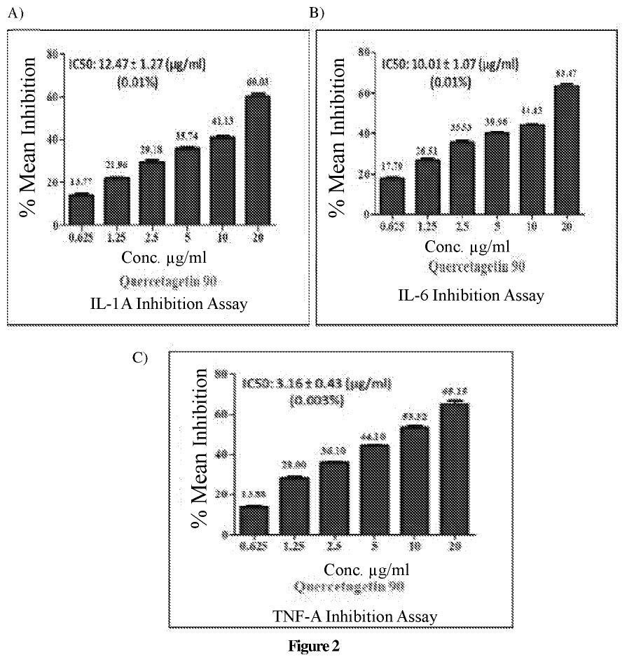

The Cellular Anti-inflammatory Assay is a cell-based assay designed for measuring the LPS (Lipo Polysaccharide) induced release of cytokines IL-1a, IL-6 and TNF-a. THP-1, and promonocytic cells are pre-treated with various concentrations of test samples followed by LPS stimulation. The cell supernatant is then used for ELISA to evaluate cytokine release. Inhibitory effect of composition with QG90 extract against LPS induced IL-6, IL-1A & TNF-alpha release in THP-1 cells was conducted. More than 60% inhibition for all 3 cytokines at 20 μg/ml was observed (FIG. 2).

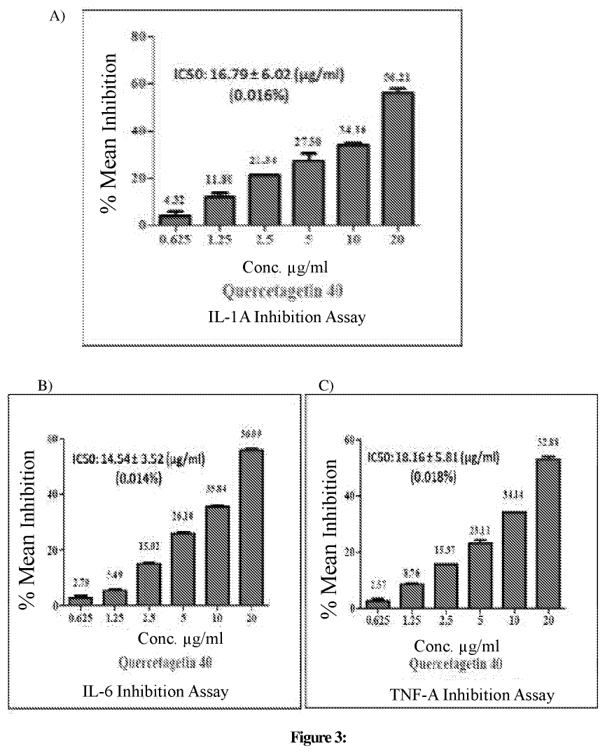

Inhibitory effect of composition with QG40 extract against LPS induced IL-6, IL-1A & TNF-alpha release in THP-1 cells was conducted. More than 50% inhibition for all 3 cytokines at 20 μg/ml was observed (FIG. 3).

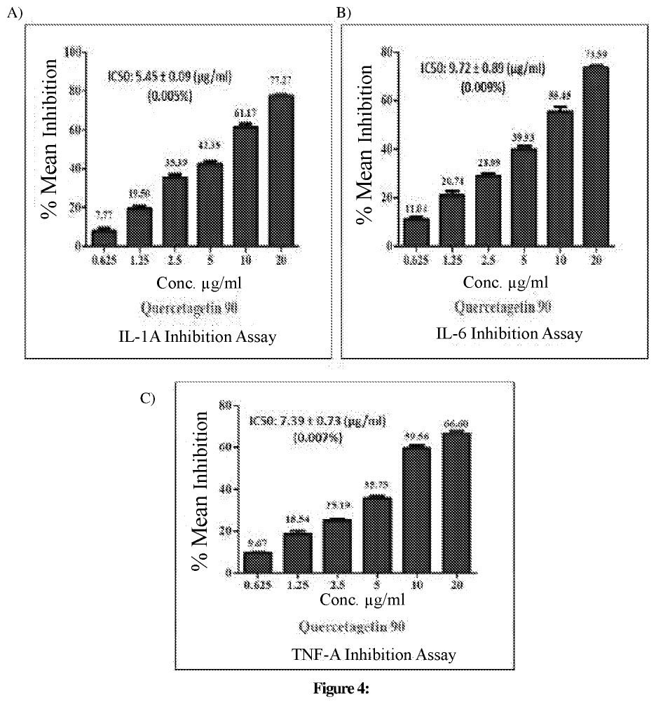

Inhibitory effect of composition with QG90 extract against Ultraviolet Radiation (UVR) induced IL-6, IL-1A & TNF-alpha release in THP-1 cells was conducted. More than 65% inhibition for all 3 cytokines at 20 μg/ml was observed (FIG. 4).

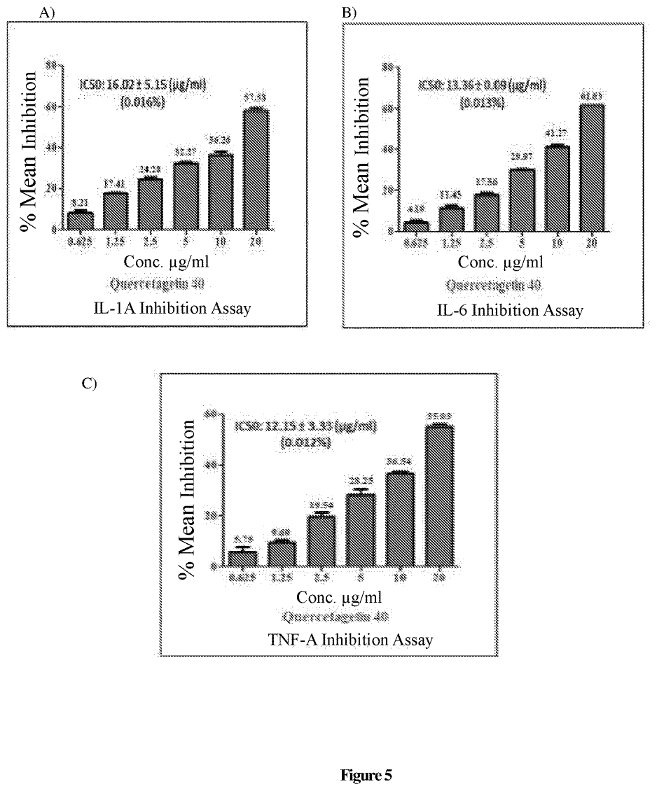

Inhibitory effect of composition with QG40 extract against Ultraviolet Radiation (UVR) induced IL-6, IL-1A & TNF-alpha release in THP-1 cells was conducted. More than 55% inhibition for all 3 cytokines at 20 μg/ml was observed, (FIG. 5).

Observation: The topical compositions comprising QG40 and QG90 extract showed better antioxidant property compared to compositions having quercetin in in vitro studies. The topical compositions comprising marigold extract enriched with quercetagetin was found to be a good UV radiation response modulator and could help in maintaining even skin tone.

Further evaluations were performed to assess the skin brightening and skin maintenance of even skin tone. It was observed, by the present inventors, that the disclosed quercetagetin enriched marigold extract was capable of anti-acne activity, anti-sebum activity and anti-inflammatory activity. The following experimental studies demonstrate the cosmetic and dermatological aspects of the disclosed marigold extract and compositions comprising the same.

### Cellular Melanin Inhibition Assay

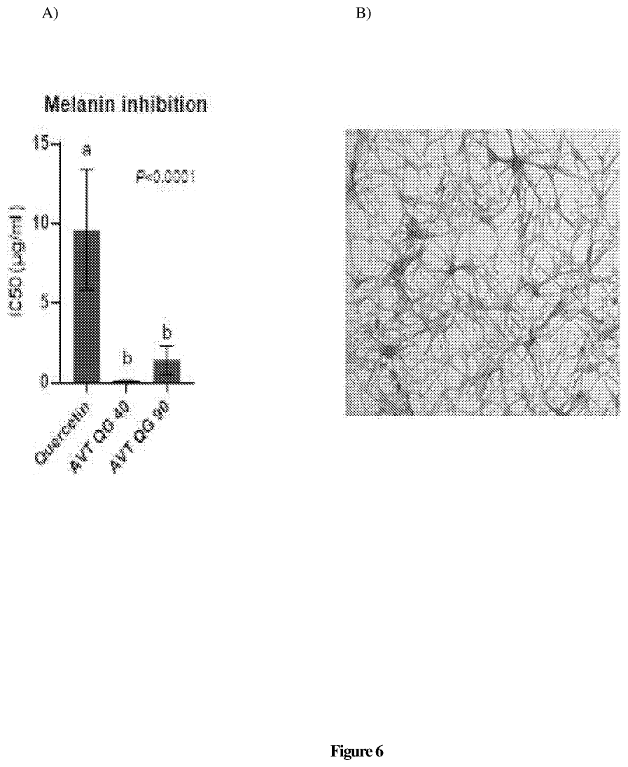

Cellular melanin inhibition assay is a cell-based assay designed for measuring Melanin inhibition in Human Adult Epidermal Melanocytes (HAEM). Darkly pigmented melanocytes were treated with test samples and incubated for 72 hrs. Post incubation, melanin was extracted and measured at an absorbance of 490 nm to evaluate the non-cytotoxic concentration of test sample (i.e. composition with QG40 extract; the composition with QG90 extract and composition with quercetin) for melanin inhibition. The results are shown in below Table 6 and FIG. 6.

TABLE 6

| Sample | IC50 (μg/ml) |
|---|---|
| Composition having QG90 extract | <2 |
| Composition having QG40 extract | <0.2 |
| Composition having Quercetin | 9.62 |
| Kojic acid (Positive control) | 25 |

The above table shows that, both, compositions having QG90 extract and QG40 extract are potent melanin inhibitors compared to Quercetin & Kojic acid in the order of quercetagetin 40>quercetagetin 90>Quercetin>Kojic Acid.

Thus, the topical composition comprising the disclosed marigold extract is effective in melanin/Tyrosinase activity inhibition.

### PIPT & HRIPT Study:

The PIPT (Primary irritation patch test) & HRIPT (Human Repeat Insult Patch Test), aims to determine the skin irritation & sensitization potential of test material by single or repeated application of occlusive patches. These tests were carried out on volunteers within the framework of national and international standards, European Union directives and current legal regulations and under the supervision of dermatologists. These tests are sometimes applied on people with severe allergies or dry skin. Irritants are the substances that may damage the skin. The damage will depend upon the nature, concentration, and duration of exposure. Irritation is manifested as inflammatory responses such as erythema (redness) and oedema (swelling), vesiculation and finally to an intense suppurate reaction without the involvement of immune system. In PIPT, the assessment is based on the mean score obtained at 24 hours after each patch removal during the induction phase. To calculate the mean score of observations made for assessing the investigational site for Skin Irritation as presented in Mean Score for Irritation. No irritative type response at 24 hours after patch removal was observed by dermatologist for the composition having QG90 extract and the composition QG40 extract.

In HRIPT, reaction during induction phase suggests skin irritation or pre-existing sensitization to the test material. In HRIPT, in the challenge phase, the assessment is based on the International Contact Dermatitis Research Group scale (ICDRG scale) at 24 hours after the patch removal, 48 hours after patch removal and 72 hours after the patch removal. Prominent reaction in the challenge phase suggests allergenicity to the ingredients of the test material. This test concludes that the that the disclosed compositions are safe to use as no evidence of allergy is reported in the tested volunteers. No irritative type of response was observed by the dermatologist during induction phase of 21 days, for composition having QG90 extract and the composition QG40 extract. No allergic type of response was observed by the dermatologist during challenge phase for these compositions.

### In Vivo Studies-Skin Brightening Studies for Cosmeceutical Application.

Objective: To evaluate the in-vivo efficacy and safety of the disclosed marigold extract in topical compositions. Evaluation was performed to assess the activity in maintaining even skin tone, improvement in skin radiance and skin brightening effect.

Primary objective was to evaluate and compare the in-vivo efficacy and safety of a skin care formulation in terms of even skin tone, improvement in skin radiance & skin brightening effect on healthy female subjects. Secondary objective was to evaluate and compare the in-vivo efficacy and safety of a skin care formulation in terms of reduction in melanin content for pigmented area/acne PIH, moisturization & for trans epidermal water loss on healthy female subjects.

Method: This is a comparative study, carried out as a “single blind test”. The subjects participating were not aware of the type of product being applied. The following techniques were included in the study. Twice a day application of the test product coded Products on a panel of 108 Female (36 subjects in each group) aged between 18 and 35 years old, presenting Fitzpatrick skin type III to V and having dull skin with acne spots & uneven skin tone led to the following results after T+14 days & T+28 days of product application.

Products tested: The products tested were Topical compositions in cream and gel based formats, wherein the amount of marigold extract varied from 0.3 wt %. 0.5 wt % to 1 wt %. The evaluation was performed for compositions having QG40 and QG90 extracts.

### Dermatological Evaluation for Efficacy:

The product was assessed by the dermatologist, through the grading on the face of defined clinical signs (observed by the dermatologist) and functional signs (felt by the subjects and reported to the dermatologist) at T0, T+14 days and T+28 days visit.

Results: Table 7 depicts the final improvement on percentage basis in comparison to 0^{th} day.

Table 8 are results for dermatological evaluation for efficacy, depicting significant improvement in skin even tone, i.e. final percentage improvement on 28^{th} day, on whole face treated with cream composition having 1 wt % of QG90 extract and 1 wt % of QG40 extract at both the time points.

No significant difference was found in Skin even tone and skin brightening on whole face treated with Placebo/Blank cream on average on whole panel at all given time point.

On comparisons between all 3 products, i.e. composition having 1 wt % of QG90 extract, composition having 1 wt % of QG40 extract, and placebo, significant difference is observed in favor of 1 wt % QG90 Cream & 1 wt % QG40 Cream, at T+28 days (Table 7)

TABLE 7

| | | 1% QG-90 | 1% QG-40 | 0.5% QG |
|---|---|---|---|---|
| | Placebo/Blank | Cream | Cream | Cream |
| Skin Even tone | No difference | 10.77% | 17.43% | 17.09% |
| Skin | No difference | 0.47 out | 0.75 out | 0.75 out |
| improvement | | of 10 | of 10 | of 10 |

TABLE 8

| | 1% QG-90 | | 1% QG-40 |
|---|---|---|---|
| | Cream | Placebo/Blank | Cream |
| Skin Brightening | 2.86% | No difference | No difference |
| Skin improvement | 0.18 out of 10 | No difference | No difference |

### Chromameter for Assessment of Skin Tone and Skin Brightening:

Procedure: Tristimulus colorimeters are made of a control unit and a measurement headline associated. The measurement headline takes the measurements from a zone of 8 mm in diameter and uses diffuse lighting as well as an angle of 0° (including specular component). The headline measurement has a light with a pulsed xenon arch lamp. A double beam system measures the incident light and the reflected light by means of six photocells. The used material is a Chromameter CR-400 (Minolta). The measurements were performed on six different sites for evaluating skin tone evenness & on both cheek bones for skin Brightening.

Results: Table 9 depicts L*, ITA°, Delta E parameter results for Chromameteric assessment of skin tone and skin brightening.

On comparison between all 3 products, i.e. composition having 1 wt % of QG90 extract, composition having 1 wt % of QG40 extract, and placebo, significant difference was observed in favor of 1% QG-90 Cream & 1% QG-40 Cream at T+28 days for L* & ITA° parameter, however, no significant difference observed for all products for Delta E parameter.

TABLE 9

| | 1% QG-90 Cream | Placebo/Blank | 1% QG-40 Cream |
|---|---|---|---|
| Chromametry/ | | | |
| L value | | | |
| T + 14 days | 0.60% | No difference | 0.85% |
| T + 28 days | 0.90% | No difference | 1.46% |
| Chromametry/ | | | |
| ITA value | | | |
| T + 14 days | 10.01% | No difference | 13.67% |
| T + 28 days | 14.74% | No difference | 22.62% |
| Chromametry/ | | | |
| Delta E | | | |
| T + 28 days | 8.37% | No difference | 6.08% |

### Mexameter for Melanin Measurement of Pigmented Area/Acne PIH (Post-Inflammatory Hyperpigmentation):

Procedure: Mexametry is the technique used to determine the melanin content of the skin. The probe of the Mexameter® MX 18 emits 3 specific light wavelengths. As the quantity of emitted light is defined, the quantity of light absorbed by the skin can be calculated. The apparatus used is a MX 18 (Courage and Khazaka, Germany).

The measurements were performed on one acne PIH. The site of the instrumental measurements and their location at the different points of the kinetics was strictly the most reproducible. The location was determined by a cutaneous marking on the instrumental measurement sites at T0 by calculating the distance of the spot from the nose and eyes. In order to reposition this marking accurately while the measurements were taken, a mapping of the skin's surface, for the measurement site and for each subject, was recorded in the case report form. Three measurements were carried out on the one acne PIH, for each subject and each examination time on T0, T+14 days and T+28 days visits.

Results: Table 10 depicts results for Mexameteric assessment of pigmented area/Acne PIH (Post-inflammatory hyperpigmentation).

On comparison between all 3 products, i.e. composition having 1 wt % of QG90 extract, composition having 1 wt % of QG40 extract, and placebo, significant difference was observed in favor of 1 wt % QG-90 Cream & 1 wt % QG-40 Cream at both the time points.

TABLE 10

| Mexametry | Placebo/Blank | 1% QG-90 Cream | 1% QG-40 Cream |
|---|---|---|---|
| T + 14 days | No difference | 2.60% | 2.88% |
| T + 28 days | No difference | 4.20% | 4.93% |

### Corneometer for Assessment of Skin Moisturization:

Procedure: It is a technique used to determine the level of moisture in the outer layers of the stratum corneum. This method is based on the relationship between the electrical properties of skin tissues and their moisture content. The principle of corneometry consists in passing a high frequency electric current through the skin between two electrodes. The electric field produced in the epidermis functions to the geometry and the dielectric constant of the electrodes and of the capacitance of the skin in contact with the probe. A moisturizing variation of the skin is traduced by a modification of the total capacitance of the system. The apparatus used is a CM 825 (Courage and Khazaka, Germany). The measurements were performed on both Cheek bones. At T0, T+14 days and T+28 days visits the measurements were carried out on both the cheek bones, after delicately wiping the skin with an absorbent paper.

Results: Table 11 depicts results for Corneometeric assessment of skin tone and skin moisturization.

On comparison between all 3 products, i.e. composition having 1 wt % of QG90 extract, composition having 1 wt % of QG40 extract, and placebo, significant difference was observed in favor of 1 wt % QG-90 Cream & 1 wt % QG-40 Cream at both the time points.

TABLE 11

| Corneometry | Placebo/Blank | 1% QG-90 Cream | 1% QG-40 Cream |
|---|---|---|---|
| T + 14 days | No difference | 29.44% | 28.82% |
| T + 28 days | No difference | 41.13% | 45.81% |

### Tewameter for Assessing Trans Epidermal Water Loss:

Procedure: Measurement is based on the Diffusion Law. The Tewameter® probe measures the density gradient of the water evaporation from the skin indirectly by the two pairs of sensors (temperature and relative humidity) inside the hollow cylinder. This is an open chamber measurement. The open chamber measurement method is the only method to assess the TEWL continuously without influencing its microenvironment. Using a microprocessor and two pairs of sensors in the probe it is possible to receive the information of corresponding temperature and moisture values of each TEWL-value during the complete measurement. The measurements were performed on three sites of cheek bone. At T0, T+14 days & T+28 days the measurements were carried out on cheek bone after delicately wiping the skin with an absorbent paper.

Results: Table 12 depicts results for Tewametric assessment of skin tone and skin moisturization.

On comparison between all 3 products, i.e. composition having 1 wt % of QG90 extract, composition having 1 wt % of QG40 extract, and placebo, significant difference was observed in favor of 1% QG-90 Cream & 1% QG-40 Cream at both the time points.

TABLE 12

| | | 1% QG-90 | 1% QG-40 |
|---|---|---|---|
| Tewametry/TEWL | Placebo/Blank | Cream | Cream |
| T + 14 days | No difference | 16.57% | 13.77% |
| T + 28 days | No difference | 19.21% | 20.27% |

### In Vivo Studies-Skin Anti-Acne and Sebum Control Studies:

Objective: To evaluate gel compositions for anti-acne and skin sebum control studies.

Method: Gel composition comprising 0.5 wt % of marigold extract enriched with quercetagetin; and Gel composition comprising 0.3 wt % of marigold extract enriched with quercetagetin was prepared and evaluated for reduction in acne, in a double blind, randomized, comparative study. The evaluation was performed with compositions having QG90 extracts. No. of subjects per product: 30-36; Time points: Screening, T0, T+14 days and T+28 days. Inclusion criteria: Females 18-30 years of age having mild to moderate acne and oily to mixed skin type.

### In Vivo Studies: Dermatological Evaluation of Efficacy of the Gel Compositions: Acne Severity (for Dermatological Application)

Method: Dermatologist evaluated the number of retentional lesions (closed comedos and opened comedos), papules, pustules, infiltrated lesions and cystic lesions on the face for each subject at T0, T+3 days, T+7 days, T+14 days and T+28 days. Evaluation was performed using Michaelsson Severity Index. For the whole lesions, decrease in severity index after several days of application, enables to conclude a reduction in acne after use of tested product.

Lesion size & Redness evaluation: Dermatologist identified the acne lesions (Papules, Pustules, Open Comedons & Closed Comedons) on face and assess them for type, redness using the Acne lesion assessment scale at T0, T+3 days, T+7 days, T+14 days & T+28 days. The rating was given in terms of a score from 1 to 5 for each studied descriptor. Decrease in score for both size and redness after several days of application, enables to conclude a reduction in acne severity by the tested product.

Results: Table 13 depicts the reduction in acne (Michaelsson Severity Index) for 0.5 wt % QG90 gel composition, and 0.3 wt % QG90 gel composition, at T0, T+3 days, T+7 days, T+14 days & T+28 days, wherein \: Significant reduction.

Significant decrease in acne severity as observed at all timepoints for, both, gel composition comprising 0.5 wt % of QG90 extract, and gel composition comprising 0.3 wt % of QG40 extract.

TABLE 13
Acne severity data

Reduction in acne (Michaelsson Severity Index)

| | 0.5% QG-90 gel | 0.3% QG gel |
|---|---|---|
| T + 3 days | ↓10.65% | No Change |
| T + 7 days | ↓ 18.63% | ↓16.94% |
| T + 14 days | ↓ 35.74% | ↓24.59% |
| T + 28 days | ↓ 43.73% | ↓ 41.40% |

Table 14 depicts the reduction in skin redness for Papules & Pustules for 0.5 wt % QG90 gel composition and 0.3 wt % QG90 gel composition, compared to baseline (Fights inflammation and Redness), at T0, T+3 days, T+7 days, T+14 days & T+28 days, wherein ⬇: Significant reduction.

No significant difference in skin redness of closed and open comedons was observed at any of study timepoint for all products.

TABLE 14
Lesion size & Redness evaluation:

| | 0.5% QG-90 gel | 0.3% QG gel |
|---|---|---|
| Papules: | | |
| Skin Redness | | |
| T + 3 days | No difference | ↓35.24% |
| T + 7 days | ↓ 16.33% | ↓ 66.67% |
| T + 14 days | ↓ 48.98% | ↓ 66.67% |
| T + 28 days | ↓ 65.31% | ↓ 66.67% |
| Pustules: | | |
| Skin Redness | | |
| T + 3 days | No Change | ↓35.24% |
| T + 7 days | No Change | ↓ 66.67% |
| T + 14 days | ↓ 45.24% | ↓ 66.67% |
| T + 28 days | ↓ 61.90% | ↓ 66.67% |
| Papules: | | |
| Lesion size | | |
| T + 3 days | No difference | ↓25.23% |
| T + 7 days | No difference | ↓ 42.99% |
| T + 14 days | ↓ 46.81% | ↓ 57.72% |
| T + 28 days | ↓ 63.83% | ↓ 68.31% |
| Pustules: | | |
| Lesion size | | |
| T + 3 days | No Change | ↓40.00% |
| T + 7 days | No Change | ↓ 52.50% |
| T + 14 days | ↓ 35.90% | ↓ 55.33% |
| T + 28 days | ↓ 51.28% | ↓ 55.33% |
| Open Comedons: | | |
| Lesion size | | |
| T + 3 days | No change | ↓ 14.29% |
| T + 7 days | | ↓38.57% |
| T + 14 days | | ↓ 50.00% |
| T + 28 days | ↓37.24% | ↓ 50.00% |
| Closed Comedons: | | |
| Lesion size | | |
| T + 3 days | No Change | ↓18.57% |
| T + 7 days | | ↓ 32.86% |
| T + 14 days | | ↓ 50.00% |
| T + 28 days | ↓ 33.33% | ↓ 50.00% |

### Sebumetry, Sebum Reduction at T Zone:

Method: The sebum measurement on the skin was based on the internationally recognized SEBUMETER® 815 method. It is a direct measurement of the sebum secretion on the skin. The significant decrease in the sebum level shows an effect of the product in terms of anti-sebum effect.

Results: Table 15 depicts the reduction in sebum content for 0.5 wt % QG90 gel composition and 0.3 wt % QG90 gel composition, at T0, T+3 days, T+7 days, T+14 days & T+28 days, wherein \: Significant reduction.

Significant Reduction in Sebum Content in both products observed at all timepoints.

TABLE 15
Reduction in Sebum Content:

| Sebum content | 0.5% QG-90 gel | 0.3% QG gel |
|---|---|---|
| T + 3 days | ↓20.61% | ↓9.51% |
| T + 7 days | ↓ 31.90% | ↓20.79% |
| T + 14 days | ↓ 37.21% | ↓ 27.44% |
| T + 28 days | ↓ 47.28% | ↓ 46.78% |

### Subject Self Evaluation:

Method: The subjects were asked to answer self-evaluation questionnaire at T+3 days, T+7 days, T+14 days & T+28 days in order to evaluate the overall opinion and their attitude towards the efficacy & safety characteristics of product under test.

Results: Products were well appreciated for efficacy, not producing additional Comedons/acne/pimples on the face up to 28 days by 100% of panel. Products well accepted for safety, no occurrence of Erythema, Oedema, Dryness, Scaling, Peeling, Itching & Tingling at any given time point during clinical study.

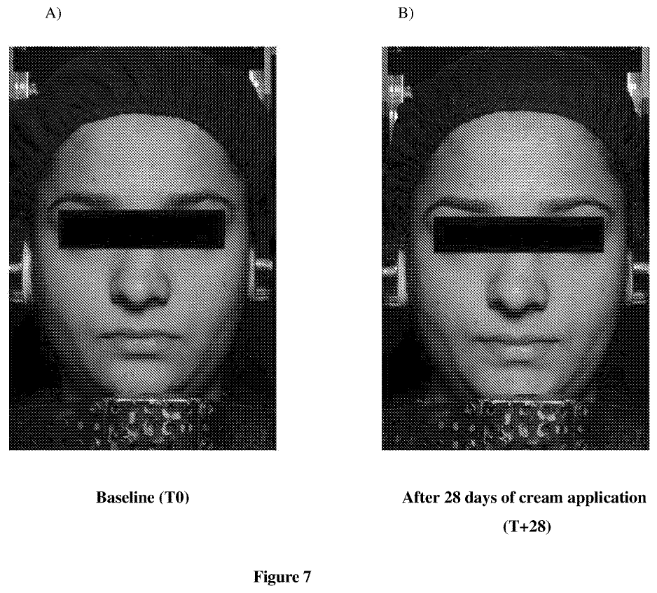

FIG. 7 are illustrative images of the whole face and ¾th under diffuse light: This technique consists of obtaining high resolution photographs of the whole face completely reproducible lighting conditions, in diffused light for illustration. FIG. 7 illustrates the effect of composition comprising QG90 extract on the skin tone improvement over 28 days' time.

### Skin Atopic Dermatitis (AD) Study:

Methods: A double-blind, randomized, three-Arm, parallel group, proof of concept study to evaluate the safety and efficacy of test product in adult human subjects with mild to moderate atopic dermatitis. Number of subjects per product were 8 to 10 subjects; Time points of screening was T0, T+14, T+30, T+45 and T+60 days; Inclusion criteria: Healthy males/Females 18-60 years of age having mild to moderate Atopic Dermatitis. The test product was cream composition comprising 1 wt % of marigold extract enriched with quercetagetin (QG90 extract).

### Dermatological Evaluation, Efficacy was Assessed in Respect of the Following Aspects:

Severity of AD: Assessment of severity of atopic dermatitis (AD) was performed using investigator global assessment (IGA) lesion size & redness evaluation.

Results: Table 16 depicts the results of severity using IGA for cream composition comprising 1 wt % of QG90 extract. The composition was found to be effective in significantly reducing the severity of atopic dermatitis when assessed by Investigator's Global Assessment (IGA) Scale.

TABLE 16

| Investigator global | |
|---|---|
| assessment (IGA) scale | 1% QG-90 Cream |
| T + 14 days | No change |
| T + 30 days | No change |
| T + 45 days | significant change (47.6%) |
| T + 60 days | significant change (66.7%) |

Skin eczema: Assessment of eczema was performed using Eczema Area and Severity Index (EASI).

Results: Table 17 depicts the results of Eczema Area and Severity Index (EASI) for cream composition comprising 1 wt % of QG90 extract. The composition was effective in significantly reducing the eczema when assessed by Eczema Area and Severity Index (EASI).

TABLE 17

| Eczema Area and Severity Index (EASI) | 1% QG-90 Cream |
|---|---|
| T + 14 days | 38.58% |
| T + 30 days | 61.19% |
| T + 45 days | 73.29% |
| T + 60 days | 85.05% |

Skin itchiness: Assessment of skin itchiness was performed using Itch Numerical Rating Scale (Itch-NRS).

Results: Table 18 depicts the reduction in the skin's itchiness when assessed by Itch NRS Scale, for cream composition comprising 1 wt % of QG90 extract. The product was found to be effective in significantly reducing the skin's itchiness when assessed by Itch NRS Scale.

TABLE 18

| skin's itchiness using Itch NRS | 1% QG-90 Cream |
|---|---|
| T + 14 days | 42.40% |
| T + 30 days | 70.00% |
| T + 45 days | 90.00% |
| T + 60 days | 100.00% |

Skin erythema and desquamation: Assessment of skin redness was performed using 5-point skin redness scale. Assessment of skin desquamation was performed using 5-point ordinal scale.

Results: Table 19 depicts the reduction in skin redness as assessed by 5-point skin redness scale, for cream composition comprising 1 wt % of QG90 extract. The composition was found to be effective in significantly reducing skin redness as assessed by 5-point skin redness scale.

TABLE 19

| Skin Erythema/Redness | 1% QG-90 Cream |
|---|---|
| T + 14 days | 61.90% |
| T + 30 days | 95.20% |
| T + 45 days | 100.00% |
| T + 60 days | 100.00% |

Table 20 depicts the reduction in the skin squamation by 5-point ordinal scale, for cream composition comprising 1 wt % of QG90 extract. The results clinically indicates that the product was effective in significantly reducing the skin desquamation as assessed by 5-point ordinal scale.

TABLE 20

| Skin Desquamation | 1% QG-90 Cream |
|---|---|
| T + 14 days | No change |
| T + 30 days | 81.00% |
| T + 45 days | 100.00% |
| T + 60 days | 100.00% |

Skin lichenification: Assessment of skin lichenification was performed using 4-point grading scale.

Results: Table 21 depicts the reduction in skin lichenification assessed using 4-point grading scale for cream composition comprising 1 wt % of QG90 extract. The results clinically indicates that the product was effective in significantly reducing the skin lichenification as assessed by 4-point grading scale.

TABLE 21

| Skin Lichenification | 1% QG-90 Cream |
|---|---|
| T + 14 days | No change |
| T + 30 days | 45.20% |
| T + 45 days | 59.50% |
| T + 60 days | 85.70% |

Skin sebum: Assessment of skin sebum level/skin oiliness was assessed by Sebumeter.

Results: Table 22 depicts the reduction in skin sebum levels assessed using Sebumeter for cream composition comprising 1 wt % of QG90 extract. The results clinically indicates that the product was effective in significantly reducing the skin sebum level/skin oiliness as assessed by Sebumeter.

TABLE 22

| Skin Sebum | 1% QG-90 Cream |
|---|---|
| T + 14 days | 3.80% |
| T + 30 days | 7.70% |
| T + 45 days | 11.50% |
| T + 60 days | 16.60% |

Skin thickness: Assessment of skin thickness was assessed by ultrasonography.

Results: Table 23 depicts the reduction in skin thickness assessed using ultrasonography for cream composition comprising 1 wt % of QG90 extract. The results clinically indicates that the product was effective in significantly reducing the skin thickness.

TABLE 23

| Skin thickness | 1% QG-90 Cream |
|---|---|
| T + 30 days | No change |
| T + 60 days | 38.92% |

Subject satisfaction and product response index data: After using the test products for 15 days, 30 days, 45 days & 60 days, the study subjects were given a questionnaire to provide feedback on the product and product response. From subject feedback, the following observations were made:
All study subjects (100%) agree that the test product helped in reducing the severity of eczema, skin itchiness and skin redness.
All study subjects (100%) agree that the test product helped in reducing/controlling the skin sebum level.
All study subjects (100%) agree that the test product helped in improving skin's moisturization.
All study subjects (100%) agree that the test product helped in retaining/improving skin's moisture by retaining/improving skin barrier function.
None of the study subjects reported feeling of any hypersensitivity reactions such as redness, swelling, dryness, burning, rash, and irritation after using test product.

### Advantages:

The present inventors have observed that the disclosed marigold extract comprising quercetagetin, and compositions thereof, are cosmeceutically and dermatologically effective. Embodiments herein are capable of decreasing sebum secretion, reducing inflammation, improving skin moisturization, controlling trans epidermal water loss, eliciting 5α-reductase inhibition, anti-acne activity and/or skin brightening activity at the target site. The compositions, as disclosed herein show improved antioxidant property, anti-inflammatory activity and immunomodulatory activity, as compared to known compounds such as quercetin. Embodiments herein are good UV radiation response modulator and capable of maintaining even skin tone. It is observed that the embodiments herein are capable of inhibiting melanin and, hence, capable of brightening the skin tone. Commercially available pure quercetagetin is an expensive raw material, embodiments herein achieve an economical and convenient method for producing extracts comprising quercetagetin in high yield and purity. Hence, the method of preparing the quercetagetin enriched marigold extract, as disclosed herein, is a scalable, convenient and efficient method. The marigold extracts obtained by the method disclosed herein may be formulated into various topically administrable cosmeceutical and dermatological compositions.

1. (canceled)

2. A method for preparing a marigold extract, said method comprising:
(a) subjecting marigold biomass to extraction using an alcohol selected from ethanol, methanol, or combination thereof, to obtain a miscella fraction;
(b) evaporating the miscella fraction to obtain a first extract;
(c) treating the first extract with a solvent comprising n-hexane to obtain a residue; and
(d) drying the residue to obtain the marigold extract, wherein the marigold extract comprises quercetagetin.

3. The method as claimed in claim 2, wherein the marigold extract comprises quercetagetin in the range of 40-75 wt % in respect of the extract.

4. The method as claimed in claim 2, said method comprising:
(a) subjecting marigold biomass to extraction using an alcohol selected from ethanol, methanol, or combination thereof, to obtain a miscella fraction;
(b) evaporating the miscella fraction to obtain a first extract;
(c) treating the first extract with a solvent comprising n-hexane to obtain a residue;
(d) drying the residue to obtain a second extract; and
(e) enriching by treating the second extract with ethanol and activated carbon, to obtain the marigold extract.

5. The method as claimed in claim 2, wherein the marigold biomass and alcohol in step (a) is in a weight ratio in the range of 1:3 to 1:5.

6. The method as claimed in claim 2, wherein the first extract of step (b) comprises quercetagetin in the range of at least 40-60 wt %.

7. The method as claimed in claim 2, wherein the ratio of first extract and solvent in step (c) is in a weight ratio in the range of 1:3 to 1:5.

8. The method as claimed in claim 4, wherein the second extract of step (d) comprises quercetagetin in the range of at least 60-75 wt %.

9. The method as claimed in claim 4, wherein the marigold extract of step (f) comprises quercetagetin in an amount of at least 90 wt %.

10. The method as claimed in claim 4, wherein second extract and ethanol in step (e) is in a weight ratio in the range of 1:30 to 1:50.

11. The method as claimed in claim 2, wherein said marigold biomass comprises water content in an amount in the range of 60-65 wt %.

12. The method as claimed in claim 4, wherein said enriching in step (e) comprises mixing the second extract with ethanol at a temperature in the range of 55-70° C., preferably 60-65° C.; adding the activated carbon; stirring for a period of 20 to 24 hrs at 20 to 27 degree temperature; and filtering to remove the activated carbon and distilling to remove the ethanol.

13. The method as claimed in claim 2, wherein the marigold biomass is obtained by dehydrating marigold flower particles by treating with dimethyl ether.

14. A marigold extract obtained by a method as claimed in claim 2, for cosmetic and dermatologic treatment of skin, said marigold extract comprising quercetagetin in an amount ranging from at least 40 wt % to 99 wt %; patuletin in an amount ranging from 1 wt % to 7 wt %; 6-Hydroxykaempherol in an amount of 0 to 4 wt %; and phytosterols in an amount ranging from 0 to 2.5 wt %, preferably 0.05 wt % to 2.5 wt %, or 0.35 wt % to 0.1 wt %.

15. The marigold extract as claimed in claim 14, further comprising galangin in an amount ranging from 0.001 wt % to 0.005 wt %, wherein said extract comprises moisture in an amount ranging from 2.5 to 5 wt %, preferably 3 to 4.5 wt %, wherein said extract comprises a nutrient selected from carbohydrate, protein, sugar, fat, crude fibre, or combination thereof, preferably in an amount ranging from 0.1 wt % to 40 wt %.

16. (canceled)

17. (canceled)

18. The marigold extract as claimed in claim 14, said extract is having 5α-reductase inhibitory activity, anti-inflammatory activity, anti-acne property and/or skin brightening property.

19. The marigold extract as claimed in claim 14, wherein said extract on application to skin tissue is capable of decreasing sebum secretion, reducing inflammation, and control trans epidermal water loss of skin tissue.

20. A topical composition for cosmetic and dermatologic treatment of skin, said composition comprising:
(a) the marigold extract obtained by a process as claimed in claim 2, in an amount ranging from 0.1 wt % to 99 wt %, preferably 0.1 wt % to 5 wt %, in respect of the topical composition; and
(b) at least one cosmetically acceptable carrier.

21. (canceled)

22. A method for preparing a topical composition, said method comprising: blending the marigold extract obtained by the method as claimed in claim 2; and a cosmetically acceptable carrier to obtain the topical composition.

23. A cosmetic method comprising applying the topical composition as claimed in claim 20 on a target site on the skin to elicit skin brightening activity, decrease sebum secretion, reduce inflammation, improve skin moisturization and control trans epidermal water loss at the target site.

24. A dermatalogical method for treatment of skin, comprising administering, topically, the topical composition as claimed in claim 20 on a target site on the skin to elicit 5α-reductase inhibition, anti-inflammatory activity, anti-acne property and/or skin brightening activity at the target site.

25. (canceled)
